(19) United States
(12) Patent Application Publication
(10) Pub. No.: US 2024/0207286 A1
(43) Pub. Date: Jun. 27, 2024

(54) ASBT INHIBITORS IN THE TREATMENT OF RENAL DISEASES

(71) Applicant: Albireo AB, Göteborg (SE)

(72) Inventors: Erik LINDSTRÖM, Göteborg (SE); Ellen STRÄNGBERG, Göteborg (SE); Jan HENGSTLER, Dortmund (DE); Ahmed GHALLAB, Dortmund (DE)

(21) Appl. No.: 18/535,313

(22) Filed: Dec. 11, 2023

(30) Foreign Application Priority Data
Dec. 9, 2022 (SE) 2251441-8

(51) Int. Cl. A61K 31/554; A61K 31/4995; A61K 31/7042; A61P 1/16

(52) U.S. Cl. CPC A61K 31/554; A61K 31/4995; A61K 31/7042; A61P 1/16

(57) ABSTRACT

The invention relates to an apical sodium-dependent bile acid transport (ASBT) inhibitor for use in the treatment of renal diseases and disorders, such as cholemic nephropathy. Such treatment can include reducing serum bile acid concentrations, increasing urinary bile acids and improving liver as well as renal parameters.

### CROSS-REFERENCE TO RELATED APPLICATIONS

This application claims priority to Swedish patent application No. 2251441-8, filed Dec. 9, 2022, the disclosure of which is incorporated herein by reference in its entirety.

### TECHNICAL FIELD

The invention relates to an apical sodium-dependent bile acid transport (ASBT) inhibitor for use in the treatment of renal diseases and disorders, such as cholemic nephropathy. Such treatment can include reducing serum bile acid concentrations, increasing urinary bile acids and improving liver as well as renal parameters.

### BACKGROUND

Cholemic nephropathy is a state of kidney injury/failure in patients with obstructive jaundice. Also referred to as bile cast nephropathy, bile acid nephropathy, icteric nephrosis/nephropathy or jaundice-related nephropathy, cholemic nephropathy represents an underestimated but important cause of renal dysfunction in cholestasis or advanced liver diseases with jaundice. It is a common complication in patients with liver diseases such as liver cirrhosis, alcoholic steatohepatitis, drug-induced cholestatic liver injury and fulminant hepatitis, and is associated with high morbidity and mortality. Cholemic nephropathy is characterized by hemodynamic changes in the liver, kidney, systemic circulation, intratubular cast formation, and tubular epithelial cell injury, but the underlying pathophysiological mechanisms are still insufficiently understood.

Toxic bile acids have been suggested to play a role in the development of kidney injury in cholestasis (Fickert et al., Hepatology 2013, vol. 58, p. 2056-2069; Krones et al., Dig. Dis. 2015, vol. 33, p. 367-375; Tinti et al., Life 2021, vol. 11, 1200). The less toxic bile acid nor-ursodeoxycholic acid was shown to ameliorate kidney injury, and has been suggested as medical treatment for cholemic nephropathy (Krones et al., J Hepatol. 2017, vol. 67, p. 110-119).

Despite an increasing interest in cholemic nephropathy and a growing understanding of the mechanism leading to this disease, there currently is no specific treatment available for this condition.

### BRIEF DESCRIPTION OF THE DRAWINGS

FIGS. 1A-1D show plots of the blood chemistry analyses in mice during 12 weeks following bile duct ligation (BDL) or sham surgery. FIG. 1A: alanine transaminase (ALT) levels; FIG. 1B: aspartate transferase (AST) levels; FIG. 1C: alkaline phosphatase (ALP) levels; FIG. 1D: total bilirubin levels.

FIGS. 2A-2D show plots of the bile acid transporter expression during 12 weeks following BDL or sham surgery. FIG. 2A: sinusoidal uptake transporter NTCP; FIG. 2B: sinusoidal uptake transporter Cyp7a1; FIG. 2C: apical transporter Bsep; FIG. 2D: sinusoidal export transporter MRP4.

FIGS. 3A-3E show plots of the expression of the apical uptake transporters ASBT (FIG. 3A) and OATP1a1 (FIG. 3B), the apical export transporter MRP4 (FIG. 3C) and the basolateral export transporters MRP3 (FIG. 3D) and OSTα (FIG. 3E) during 12 weeks following BDL or sham surgery.

FIG. 4 shows a plot of the total concentration of bile acids in urine following treatment of BDL mice with different doses of Compound 1.

FIGS. 5A-5C show plots of the concentrations of individual bile acids in urine following treatment of BDL mice with different doses of Compound 1. FIG. 5A: tauro-α/β-muricholate; FIG. 5B: taurocholate; FIG. 5C: taurocholic acid sulfate.

FIG. 6 shows a plot of the total concentration of bile acids in urine following treatment of mice (BDL or sham) with vehicle or Compound 1 for 6 weeks.

FIG. 7 are plots of the concentrations of individual bile acids (ω-muricholate, tauro-ω-muricholate, tauro-α/β-muricholate and β-muricholate) in urine following treatment of mice (BDL or sham) with vehicle or Compound 1 for 6 weeks.

FIG. 8 are plots of the concentrations of individual bile acids (glycocholate, tauroursodeoxycholate, taurocholate and cholate) in urine following treatment of mice (BDL or sham) with vehicle or Compound 1 for 6 weeks.

FIG. 9 are plots of the concentrations of individual bile acids (ursodeoxycholate, hyodeoxycholate, taurochenodeoxycholate and taurodeoxycholate) in urine following treatment of mice (BDL or sham) with vehicle or Compound 1 for 6 weeks.

FIG. 10 are plots of the concentrations of individual bile acids (chenodeoxycholate, deoxycholate, taurolitocholate and taurocholic acid sulfate) in urine following treatment of mice (BDL or sham) with vehicle or Compound 1 for 6 weeks.

FIG. 11 is a plot of the change in weight of mice (BDL or sham) during treatment with vehicle or Compound 1 for 6 weeks.

FIG. 12 is a plot of the survival of mice (BDL or sham) during treatment with vehicle or Compound 1 for 6 weeks.

FIG. 13 is a plot of the concentration of the urinary biomarker neutrophil gelatinase-associated lipocalin (NGAL) during treatment of mice (BDL or sham) with vehicle or Compound 1 for 6 weeks.

FIG. 14 is a plot of the concentration of the urinary biomarker kidney injury molecule-1 (KIM-1) during treatment of mice (BDL or sham) with vehicle or Compound 1 for 6 weeks.

FIGS. 15A-15E show plots of the blood chemistry analyses of mice (BDL or sham) during treatment with vehicle or Compound 1 for 6 weeks. FIG. 15A: alanine transaminase (ALT) levels; FIG. 15B: aspartate transferase (AST) levels; FIG. 15C: alkaline phosphatase (ALP) levels; FIG. 15D: total bilirubin levels; FIG. 15E: blood urea nitrogen (BUN) levels.

FIGS. 16A-16G show plots of the expression of different bile acid transporters following treatment of mice (BDL or sham) with vehicle or Compound 1 for 6 weeks. FIG. 16A: NTCP; FIG. 16B: Bsep; FIG. 16C: OATP; FIG. 16D: MRP2; FIG. 16E: MRP3; FIG. 16F: Cyp7a1; FIG. 16G: MRP4.

FIGS. 17A-17G show plots of the expression of different bile acid transporters following treatment of mice (BDL or sham) with vehicle or Compound 1 for 6 weeks. FIG. 17A: ASBT; FIG. 17B: OATP2b1; FIG. 17C: OATP1a1; FIG. 17D: MRP2; FIG. 17E: MRP3; FIG. 17F: MRP4; FIG. 17G: OST-α.

FIGS. 18A-18D show plots of the survival rate (%) following treatment of mice (BDL or sham) with vehicle or Compound 1 for 28 days, with treatment starting at different times from BDL or sham. FIG. 18A: treatment from day 3; FIG. 18B: treatment from day 21; FIG. 18C: treatment from day 42; FIG. 18D: treatment from day 63.

FIGS. 19A-19D show plots of the weight change (%) following treatment of mice (BDL or sham) with vehicle or Compound 1 for 28 days, with treatment starting at different times from BDL or sham. FIG. 19A: treatment from day 3; FIG. 19B: treatment from day 21; FIG. 19C: treatment from day 42; FIG. 19D: treatment from day 63.

FIGS. 20A-20B show plots of the total concentration of bile acids in serum (FIG. 20A) and urine (FIG. 20B) following treatment of mice (BDL or sham) with vehicle, Compound 1 or Compound 2 for 19 days.

FIG. 21 shows a plot of the concentration of the urinary biomarker NGAL following treatment of mice (BDL or sham) with vehicle, Compound 1 or Compound 2 for 19 days. (Data are expressed as Mean±SEM).

FIGS. 22A-22B show plots of the total concentration of bile acids in serum (FIG. 22A) and urine (FIG. 22B) following treatment of BDL mice with vehicle or different ASBT inhibitors for 5 days. (Data are expressed as Mean±SEM; **p<0.01 & ****p>0.001 vs BDL control group by ONE way ANOVA followed by Dunnett's multiple comparison test; $$$$p>0.0001 vs BDL control by student's Unpaired T-test).

FIG. 23 shows a plot of the concentration of the urinary biomarker NGAL following treatment of BDL mice with vehicle or different ASBT inhibitors for 5 days. (Data are expressed as Mean±SEM; *p<0.05 vs BDL control group by ONE way ANOVA followed by Dunnett's multiple comparison test; $p>0.05 & $$p<0.01 vs BDL control by student's Unpaired T-test).

FIGS. 24A-24B show plots of the concentration of the ASBT inhibitor in serum at day 8, at 2 and 6 hours post dosing. FIG. 24B: Compound X; FIG. 24B: Compound 5. (Data are expressed as Mean±SEM).

FIG. 25 shows a plot of the concentration of Compound X and Compound 5 in urine at day 8. (Data are expressed as Mean±SEM).

### DETAILED DESCRIPTION OF THE INVENTION

The apical sodium dependent bile acid transporter (ASBT, also called ileal bile acid transporter (IBAT), ISBT, ABAT or NTCP2; gene symbol SLC10A2) is expressed in the apical membrane of ileal enterocytes, renal proximal tubular epithelial cells, biliary epithelium, large cholangiocytes and gallbladder epithelial cells. In the ileum, where ASBT is predominantly expressed, it mediates resorption of conjugated bile acids for recirculation back to the liver. Inhibition of ASBT disrupts the enterohepatic circulation and leads to fecal elimination of bile acids similar to surgical interruption of the enterohepatic circulation. The removal of bile acids from the enterohepatic circulation results in a decrease in the level of bile acids in serum and the liver. ASBT inhibitors have therefore been developed for treatment of liver diseases that are associated with elevated bile acid levels.

ASBT is also expressed in the proximal tubular epithelial cells of the kidneys. Systemically available ASBT inhibitors may therefore also inhibit the reuptake of bile acids in the kidneys. It is believed that this leads to increased levels of bile acids in urine, and to an increased removal of bile acids from the body via the urine. Targeting renal ASBT may thus be an additional means of increasing bile acid excretion, thereby further reducing bile acid load in serum and the liver.

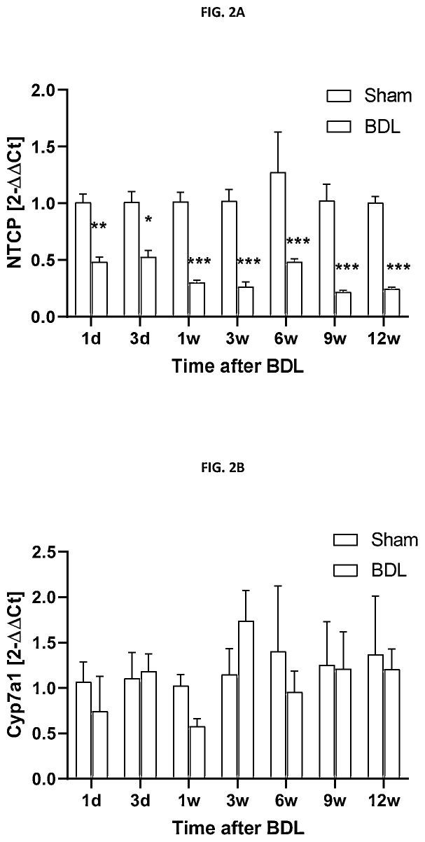

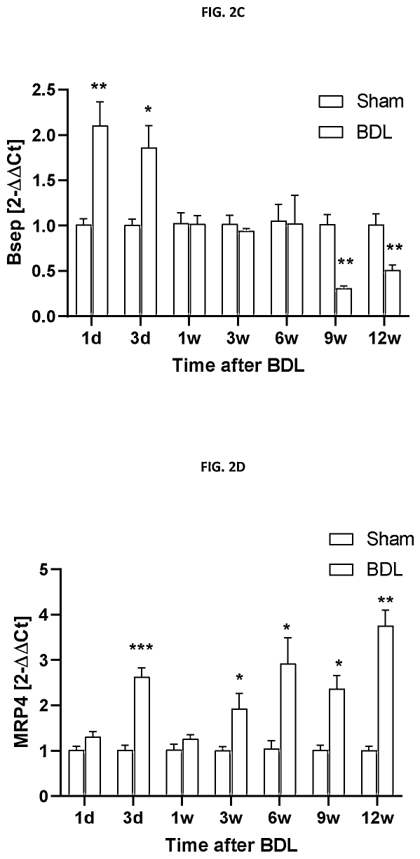

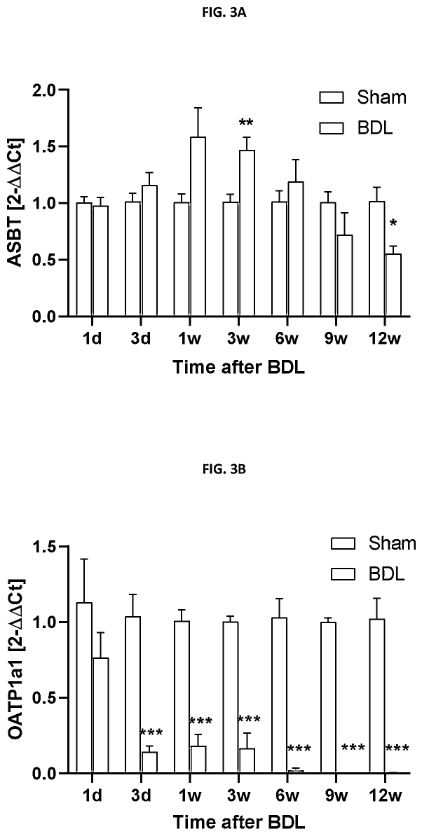

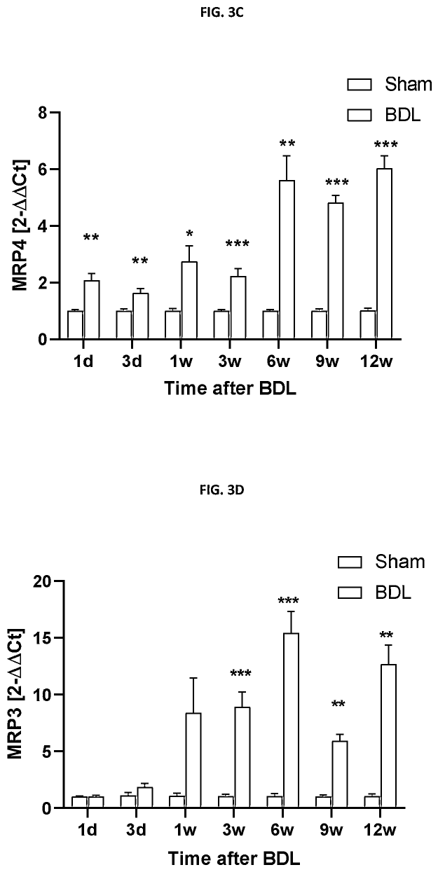

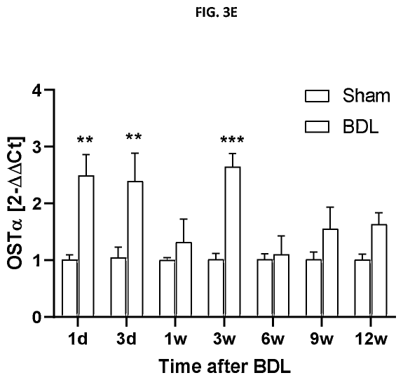

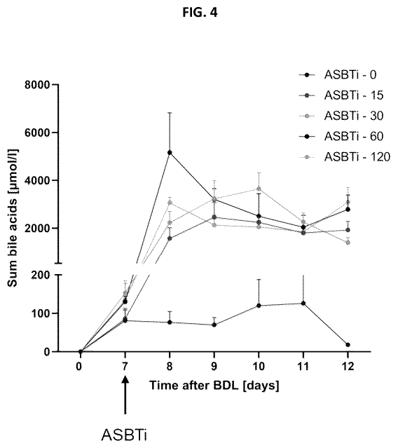

Many agents that are not cleared by the diseased liver end up in renal tissue, where they may cause renal disease. It is currently unclear by which mediators the diseased liver causes kidney injury, but candidates are bile acids, bilirubin, and inflammatory mediators such cytokines. It has now been discovered that ASBT inhibitors also may play a crucial role in mediating the toxic effects of bile acids in the kidneys. The inventors have observed that ASBT is strongly downregulated after bile duct litigation (BDL) in mice (see FIGS. 2A-2D and 3A-3E), and that inhibition of renal ASBT drastically ameliorates cholemic nephropathy in mice. In particular, it has been found that the renal proximal tubular epithelial cell is responsible for the kidney disease, as this cell accumulates bile acids by the transporter ASBT. Inhibition of ASBT was found to almost completely inhibit uptake of bile acids into the proximal tubular epithelial cell, thereby preventing bile casts and renal disease. This was unexpected, as ASBT is mainly expressed in the proximal tubuli while bile casts form in the distal tubuli. In view of the many mediators that may be involved in the cause of renal disease, it is surprising that an ASBT inhibitor has such a profound effect. The similarity of histological key features and ASBT expression in the kidneys of patients with cholemic nephropathy suggests a therapeutic perspective also in humans. Inhibition of ASBT may therefore have a protective effect on the kidneys, not least in conditions where patients also suffer from advanced liver diseases.

In a first aspect, therefore, the invention relates to an ASBT inhibitor (e.g., any of the ASBT inhibitors described herein), or a pharmaceutically acceptable salt thereof, for use in the treatment of a renal disease or disorder. In some embodiments, the renal disease or disorder is selected from the group consisting of cholemic nephropathy, chronic nephropathy, hyperbilirubinemia, renal dysfunction of obstructive jaundice, aging-induced impaired mitochondrial functions in the kidney, renal inflammation, acute kidney injury (AKI), kidney ischemia/reperfusion injury (IRI), chronic kidney disease (CKD), chronic renal insufficiency, end-stage renal disease (ESRD), proximal tubule damage in the kidney, hepatorenal syndrome type 1, hepatorenal syndrome type 2, and acute-on-chronic liver disease.

Also provided herein are methods of treating a renal disease or disorder in a subject, the method comprising administering to the subject a therapeutically effective amount of an ASBT inhibitor (e.g., any of the ASBT inhibitors described herein), or a pharmaceutically acceptable salt thereof. In some embodiments, the renal disease or disorder is selected from the group consisting of cholemic nephropathy, chronic nephropathy, hyperbilirubinemia, renal dysfunction of obstructive jaundice, aging-induced impaired mitochondrial functions in the kidney, renal inflammation, acute kidney injury (AKI), kidney ischemia/reperfusion injury (IRI), chronic kidney disease (CKD), chronic renal insufficiency, end-stage renal disease (ESRD), proximal tubule damage in the kidney, hepatorenal syndrome type 1, hepatorenal syndrome type 2, and acute-on-chronic liver disease.

In some embodiments, the renal disease or disorder is a bile acid dependent renal disease or disorder, e.g., a renal disease or disorder that may benefit from partial or full inhibition of renal ASBT. Non-limiting examples of a bile acid dependent renal disease or disorder include cholemic nephropathy, chronic nephropathy, hyperbilirubinemia, renal dysfunction of obstructive jaundice, aging-induced impaired mitochondrial functions in the kidney, renal inflammation, acute kidney injury (AKI), kidney ischemia/reperfusion injury (IRI), chronic kidney disease (CKD), chronic renal insufficiency, end-stage renal disease (ESRD), proximal tubule damage in the kidney, hepatorenal syndrome type 1, hepatorenal syndrome type 2, and acute-on-chronic liver disease.

In some embodiments, the invention relates to an ASBT inhibitor (e.g., any of the ASBT inhibitors described herein), or a pharmaceutically acceptable salt thereof, for use in the treatment of cholemic nephropathy.

ASBT inhibitors

In some embodiments, the ASBT inhibitor is a compound disclosed in, e.g., WO 93/16055, WO 94/18183, WO 94/18184, WO 96/05188, WO 96/08484, WO 96/16051, WO 97/33882, WO 98/03818, WO 98/07449, WO 98/40375, WO 99/35135, WO 99/64409, WO 99/64410, WO 00/01687, WO 00/47568, WO 00/61568, WO 00/38725, WO 00/38726, WO 00/38727, WO 00/38728, WO 00/38729, WO 01/66533, WO 01/68096, WO 02/32428, WO 02/50051, WO 03/020710, WO 03/022286, WO 03/022825, WO 03/022830, WO 03/061663, WO 03/091232, WO 03/106482, WO 2004/006899, WO 2004/076430, WO 2007/009655, WO 2007/009656, WO 2008/058628, WO 2008/058630, WO 2011/137135, WO 2019/234077, WO 2020/161216, WO 2020/161217, WO 2021/110884, WO 2021/110885, WO 2021/110886, WO 2021/110887, WO 2022/029101, DE 19825804, EP 864582, EP 489423, EP 549967, EP 573848, EP 624593, EP 624594, EP 624595, EP 624596, EP 0864582, EP 1173205, EP 1535913, EP 1719768 or EP 3210977.

In some embodiments, the ASBT inhibitor is a compound of formula (I):

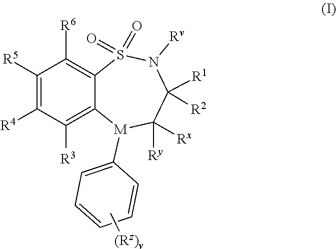

wherein:
R^{v} is selected from hydrogen or C₁₋₆alkyl;
one of R¹ and R² is selected from hydrogen, C₁₋₆alkyl or C₂₋₆alkenyl and the other is selected from C₁₋₆alkyl or C₂₋₆alkenyl;
R^{x} and R^{y} are independently selected from the group consisting of hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkyIS(O)ₐ wherein a is 0 to 2;
M is selected from —N— or —CH—;
R² is selected from the group consisting of halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;
v is 0-5;
one of R⁴ and R⁵ is a group of formula (IA):

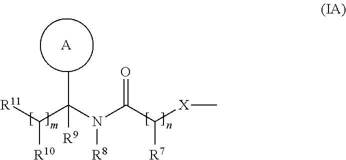

R³ and R⁶ and the other of R⁴ and R⁵ are independently selected from the group consisting of hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl) carbamoyl, N,N-(C₁₋₄alkyl)₂carbamoyl, C₁₋₄alkylS(O), wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;
X is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted by one or more substituents selected from R¹⁷;
R⁷ is hydrogen, C¹⁻⁴alkyl, carbocyclyl or heterocyclyl; wherein R⁷ is optionally substituted by one or more substituents selected from R¹⁸;
R⁸ is hydrogen or C₁₋₄alkyl;
R⁹ is hydrogen or C₁₋₄alkyl;
R¹⁰ is hydrogen, C₁₋₄alkyl, carbocyclyl or heterocyclyl; wherein R¹⁰ is optionally substituted by one or more substituents selected from R¹⁹;
R¹¹ is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆alkyl; or R¹¹ is a group of formula (IB) or (IC):

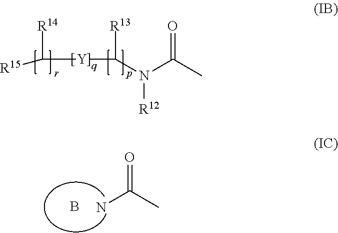

wherein:
Y is —N(Rⁿ)—, —N(Rⁿ)C(O)—, —N(Rⁿ)C(O)(CR^{s}R^{t})ᵥN(Rⁿ)C(O)—, —O—, and —S(O)a—; wherein a is 0-2, v is 1-2, R^{s} and R^{t} are independently selected from hydrogen or C₁₋₄alkyl optionally substituted by R²⁶ and Rⁿ is hydrogen or C₁₋₄alkyl;
R¹² is hydrogen or C₁₋₄alkyl;
R¹³ and R¹⁴ are independently selected from hydrogen, C₁₋₆alkyl, carbocyclyl or heterocyclyl; and when q is 0, R¹⁴ may additionally be selected from hydroxy; wherein R¹³ and R¹⁴ may be independently optionally substituted by one or more substituents selected from R²⁰;
R¹⁵ is carboxy, sulpho, sulphino, phosphono, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from C₁₋₆alkyl;
p is 1-3; wherein the values of R¹³ may be the same or different;
q is 0-1;
r is 0-3; wherein the values of R¹⁴ may be the same or different;
m is 0-2; wherein the values of R¹⁰ may be the same or different;
n is 1-3; wherein the values of R⁷ may be the same or different;
Ring B is a nitrogen linked heterocyclyl substituted on carbon by one group selected from R²³, and optionally additionally substituted on carbon by one or more R²⁴; and wherein if said nitrogen linked heterocyclyl contains an —NH— moiety, that nitrogen may be optionally substituted by a group selected from R²⁵;
R¹⁶, R¹⁷ and R¹⁸ are independently selected from the group consisting of halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N, N-(C₁₋₄alkyl)₂amino, C₁₋₄alkanoylamino, N-(C₁₋₄alkyl)carbamoyl, N, N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;
R¹⁹, R²⁰, R²⁴ and R²⁶ are independently selected from the group consisting of halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, benzyloxycarbonylamino, (C₁₋₄alkyl)₃silyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹, R²⁰, R²⁴ and R²⁶ may be independently optionally substituted on carbon by one or more R²²;
R²¹ and R²² are independently selected from the group consisting of halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
R²³ is carboxy, sulpho, sulphino, phosphono, —P(O)(OR^{g})(OR^{h}), —P(O)(OH)(OR^{g}), —P(O)(OH)(R^{g}) or —P(O)(OR_{g}) (Rₕ) wherein R^{g} and R^{h} are independently selected from C₁₋₆alkyl; and
R²⁵ is selected from the group consisting of C₁₋₆alkyl, C₁₋₆alkanoyl, C₁₋₆alkylsulphonyl, C₁₋₆alkoxycarbonyl, carbamoyl, N-(C₁₋₆alkyl) carbamoyl, N, N-(C₁₋₆alkyl)carbamoyl, benzyl, benzyloxycarbonyl, benzoyl and phenylsulphonyl;

or a pharmaceutically acceptable salt thereof.

In some embodiments, the ASBT inhibitor is a compound of formula (II):

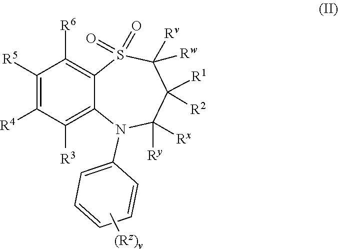

wherein:
R^{v} and R^{w} are independently selected from hydrogen or C₁₋₆alkyl;
R¹ and R² are independently selected from C₁₋₆alkyl;
R^{x} and R^{y} are independently selected from hydrogen or C₁₋₆alkyl, or one of R^{x} and R^{y} is hydrogen or C₁₋₆alkyl and the other is hydroxy or C₁₋₆alkoxy;
R^{z} is selected from the group consisting of halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, ureido, N′-(C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂-N-(C₁₋₆alkyl)ureido, N-(C₁₋₆alkyl)sulphamoyl and N,N-(C₁₋₆alkyl)₂sulphamoyl;
v is 0-5;
one of R⁴ and R⁵ is a group of formula (IIA):

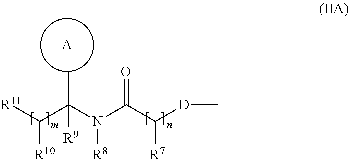

R³ and R⁶ and the other of R⁴ and R⁵ are independently selected from the group consisting of hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂-amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl;

wherein R³ and R⁶ and the other of R⁴ and R⁵ may be optionally substituted on carbon by one or more R¹⁶;
D is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted by one or more substituents selected from R₁₇;
R⁷ is hydrogen, C₁₋₄alkyl, carbocyclyl or heterocyclyl; wherein R⁷ is optionally substituted by one or more substituents selected from R¹⁸;
R⁸ is hydrogen or C₁₋₄alkyl;
R⁹ is hydrogen or C₁₋₄alkyl;
R¹⁰ is hydrogen, C₁₋₄alkyl, carbocyclyl or heterocyclyl; wherein R¹⁰ is optionally substituted by one or more substituents selected from R¹⁹;
R¹¹ is selected from the group consisting of carboxy, sulpho, sulphino, phosphono, tetrazolyl, —P(O)(OR^{c})(OR^{d}), —P(O)(OH)(OR^{c}), —P(O)(OH)(R_{d}) and —P(O)(OR^{c})(R^{d}) wherein R^{c} and R^{d} are independently selected from C₁₋₆alkyl; or R¹¹ is a group of formula (IIB):

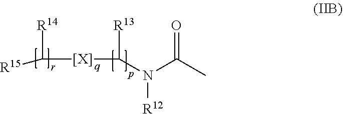

wherein:
X is —N(R^{q})—, —N(R^{q})C(O)—, —O—, or —S(O)ₐ—; wherein a is 0-2 and R^{q} is hydrogen or C₁₋₄alkyl;
R¹² is hydrogen or C₁₋₄alkyl;
R¹³ and R¹⁴ are independently selected from the group consisting of hydrogen, C₁₋₄alkyl, carbocyclyl, heterocyclyl and R²³; wherein said C₁₋₄alkyl, carbocyclyl or heterocyclyl may be independently optionally substituted by one or more substituents selected from R²⁰;
R¹⁵ is selected from the group consisting of carboxy, sulpho, sulphino, phosphono, tetrazolyl, —P(O)(O^{e})(OR^{f}), —P(O)(OH)(OR^{e}), —P(O)(OH)(R^{e}) and —P(O)(OR^{e})(R^{f}) wherein R^{e} and R^{f} are independently selected from C₁₋₆alkyl; or R¹⁵ is a group of formula (IIC):

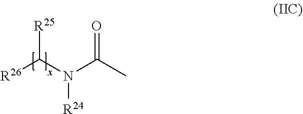

wherein:
R²⁴ is hydrogen or C₁₋₄alkyl;
R²⁵ is selected from the group consisting of hydrogen, C₁₋₄alkyl, carbocyclyl, heterocyclyl and R²⁷; wherein said C₁₋₄alkyl, carbocyclyl or heterocyclyl may be independently optionally substituted by one or more substituents selected from R²⁸;
R²⁶ is selected from the group consisting of carboxy, sulpho, sulphino, phosphono, tetrazolyl, —P(O)(OR^{g})(OR^{h}), —P(O)(OH)(OR^{g}), —P(O)(OH)(R^{g}) and —P(O)(OR^{g})(R^{h}) wherein R^{g} and R^{h} are independently selected from C₁₋₆alkyl;
p is 1-3; wherein the values of R¹³ may be the same or different;
q is 0-1;
r is 0-3; wherein the values of R¹⁴ may be the same or different;
m is 0-2; wherein the values of R¹⁰ may be the same or different;
n is 1-3; wherein the values of R⁷ may be the same or different;
z is 0-3; wherein the values of R²⁵ may be the same or different;
R¹⁶, R¹⁷ and R¹⁸ are each independently selected from the group consisting of halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl;
wherein R¹⁶, R¹⁷ and R¹⁸ may be independently optionally substituted on carbon by one or more R²¹;
R¹⁹, R²⁰, R²³, R²⁷ and R²⁸ are each independently selected from the group consisting of halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl) carbamoyl, N,N-(C₁₋₄alkyl)₂carbamoyl, C₁₋₄alkylS(O)ₐ wherein a is 0 to 2, C₁₋₄alkoxycarbonyl, N-C₁₋₄alkyl)sulphamoyl, N,N-(C₁₋₄alkyl)₂sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹, R²⁰, R²³, R²⁷ and R²⁸ may be independently optionally substituted on carbon by one or more R²²; and
R²¹ and R²² are independently selected from the group consisting of halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;

or a pharmaceutically acceptable salt thereof.

In some embodiments, the ASBT inhibitor is a compound of formula (III):

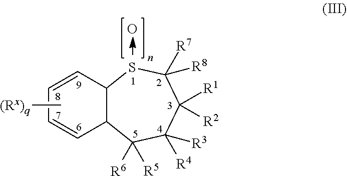

wherein:
q is an integer from 1 to 4;
n is an integer from 0 to 2;
R¹ and R² are independently selected from the group consisting of H, alkyl, alkenyl, alkynyl, haloalkyl, alkylaryl, arylalkyl, alkoxy, alkoxyalkyl, dialkylamino, alkylthio, (polyalkyl)aryl, and cycloalkyl,
wherein alkyl, alkenyl, alkynyl, haloalkyl, alkylaryl, arylalkyl, alkoxy, alkoxyalkyl, dialkylamino, alkylthio, (polyalkyl)aryl, and cycloalkyl optionally are substituted with one or more substituents selected from the group consisting of OR⁹, NR⁹R¹⁰, N⁺R⁹R¹⁰R^{w}A⁻, SR⁹, S⁺R⁹R¹⁰A⁻. P⁺R⁹R¹⁰R¹¹A⁻, S(O)R⁹, SO₂R⁹, SO₃R⁹, CO₂R⁹, CN, halogen, oxo, and CONR⁹R¹⁰,
wherein alkyl, alkenyl, alkynyl, alkylaryl, alkoxy, alkoxyalkyl, (polyalkyl)aryl, and cycloalkyl optionally have one or more carbons replaced by O, NR⁹, N⁺R⁹R¹⁰A⁻, S, SO, SO₂, S⁺R⁹A⁻, P⁺R⁹R¹⁰A⁻, or phenylene,
wherein R⁹, R¹⁰, and R^{w} are independently selected from the group consisting of H, alkyl, alkenyl, alkynyl, cycloalkyl, aryl, acyl, heterocycle, ammoniumalkyl, arylalkyl, carboxyalkyl, carboxyheteroaryl, carboxyheterocycle, carboalkoxyalkyl, carboxyalkylamino, heteroarylalkyl, heterocyclylalkyl, and alkylammoniumalkyl; or
R¹ and R² taken together with the carbon to which they are attached form C₃₋₁₀cycloalkyl;
R³ and R⁴ are independently selected from the group consisting of H, alkyl, alkenyl, alkynyl, acyloxy, aryl, heterocycle, OR⁹, NR9R¹⁰, SR3, S(O)R3, SO2R3, and SO3R3, wherein R³ and R¹⁰ are as defined above; or
R³ and R⁴ together form ═O, ═NOR¹¹, ═S, ═NNR¹¹R¹², ═NR⁹, or ═CR¹¹R¹²,
wherein R¹¹ and R¹² are independently selected from the group consisting of H, alkyl, alkenyl, alkynyl, aryl, arylalkyl, alkenylalkyl, alkynylalkyl, heterocycle, carboxyalkyl, carboalkoxyalkyl, cycloalkyl, cyanoalkyl, OR⁹, NR₉R¹⁰, SR⁹, S(O)R⁹, SO₂R⁹, SO₃R⁹, CO₂R⁹, CN, halogen, oxo, and CONR⁹R¹⁰, wherein R⁹ and R¹⁰ are as defined above, provided that both R³ and R⁴ cannot be OH, NH₂, and SH, or
R¹¹ and R¹² together with the nitrogen or carbon atom to which they are attached form a cyclic ring;
R⁵ is aryl substituted with one or more OR^{13a},
wherein R^{13a} is selected from the group consisting of alkylarylalkyl, alkylheteroarylalkyl, alkylheterocyclylalkyl, heterocyclylalkyl, heteroarylalkyl, quaternary heterocyclylalkyl, alkylammoniumalkyl, and carboxyalkylaminocarbonylalkyl,
R^{13a} is optionally substituted with one or more groups selected from the group consisting of hydroxy, amino, sulfo, carboxy, alkyl, carboxyalkyl, heterocycle, heteroaryl, sulfoalkyl, quaternary heterocycle, quaternary heteroaryl, quaternary heterocyclylalkyl, quaternary heteroarylalkyl, guanidinyl, OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R10R11A⁻, S⁺R⁹R¹⁰A⁻, and C(O)OM,
wherein A⁻ is a pharmaceutically acceptable anion and M is a pharmaceutically acceptable cation, wherein R¹⁶ and R¹⁷ are independently selected from the substituents constituting R⁹ and M; and
R⁶ is selected from the group consisting of H, alkyl, alkenyl, alkynyl, aryl, cycloalkyl, heterocycle, quaternary heterocycle, OR³⁰, SR⁹, S(O)R⁹, SO₂R⁹, and SO₃R⁹,
wherein alkyl, alkenyl, alkynyl, aryl, cycloalkyl, heterocycle, quaternary heterocycle, and quaternary heteroaryl can be substituted with one or more substituent groups independently selected from the group consisting of alkyl, alkenyl, alkynyl, polyalkyl, polyether, aryl, haloalkyl, cycloalkyl, heterocycle, arylalkyl, quaternary heterocycle, quaternary heteroaryl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, COR¹³, NR¹³C(O)R¹⁴, NR¹³C(O)NR¹⁴R¹⁵, NR¹³CO₂R¹⁴, OC(O)R¹³, OC(O)NR¹³R¹⁴, NR¹³SOR¹⁴, NR¹³SO₂R¹⁴, NR¹³SONR¹⁴R¹⁵, NR¹³SO₂NR¹⁴R¹⁵, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻, wherein:
A⁻ is a pharmaceutically acceptable anion and M is a pharmaceutically acceptable cation,
said alkyl, alkenyl, alkynyl, polyalkyl, polyether, aryl, haloalkyl, cycloalkyl, and heterocycle can be further substituted with one or more substituent groups selected from the group consisting of OR⁷, NR⁷R⁸, SR⁷, S(O)R⁷, SO₂R⁷, SO₃R⁷, CO₂R⁷, CN, oxo, CONR⁷R⁸, N⁺R⁷R⁸R⁹A⁻, alkyl, alkenyl, alkynyl, aryl, cycloalkyl, heterocycle, arylalkyl, quaternary heterocycle, quaternary heteroaryl, P(O)R⁷R⁸, P⁺R⁷R⁸R⁹A⁻, and P(O)(OR⁷)OR⁸, and
wherein said alkyl, alkenyl, alkynyl, polyalkyl, polyether, aryl, haloalkyl, cycloalkyl, and heterocycle can optionally have one or more carbons replaced by O, NR⁷, N⁺R⁷R⁸A⁻, S, SO, SO₂, S⁺R⁷A⁻, PR⁷, P(O)R⁷, P⁺R⁷R⁸A⁻, or phenylene, and R¹³, R¹⁴, and R¹⁵ are independently selected from the group consisting of hydrogen, alkyl, alkenyl, alkynyl, polyalkyl, polyether, aryl, arylalkyl, alkylarylalkyl, alkylheteroarylalkyl, alkylheterocyclylalkyl, cycloalkyl, heterocycle, heteroaryl, quaternary heterocycle, quaternary heteroaryl, heterocyclylalkyl, heteroarylalkyl, quaternary heterocyclylalkyl, quaternary heteroarylalkyl, alkylammoniumalkyl, and carboxyalkylaminocarbonylalkyl,
wherein alkyl, alkenyl, alkynyl, arylalkyl, heterocycle, and polyalkyl optionally have one or more carbons replaced by O, NR⁹, N⁺R⁹R¹⁰A⁻, S, SO, SO₂, S⁺R⁹A⁻, PR⁹, P⁺R⁹R¹⁰A⁻, P(O)R⁹, phenylene, carbohydrate, amino acid, peptide, or polypeptide, and
R¹³, R¹⁴, and R¹⁵ are optionally substituted with one or more groups selected from the group consisting of hydroxy, amino, sulfo, carboxy, alkyl, carboxyalkyl, heterocycle, heteroaryl, sulfoalkyl, quaternary heterocycle, quaternary heteroaryl, quaternary heterocyclylalkyl, quaternary heteroarylalkyl, guanidinyl, OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹⁰R¹¹A⁻, S⁺R⁹R¹⁰A⁻, and C(O)OM,
wherein R¹⁶ and R¹⁷ are independently selected from the substituents constituting R⁹ and M; or
R¹³ and R¹⁴, together with the nitrogen atom to which they are attached form a mono- or polycyclic heterocycle that is optionally substituted with one or more radicals selected from the group consisting of oxo, carboxy and quaternary salts; or
R¹⁴ and R¹⁵, together with the nitrogen atom to which they are attached, form a cyclic ring; and

R³⁰ is selected from the group consisting of alkyl, alkenyl, alkynyl, cycloalkyl, aryl, acyl, heterocycle, ammoniumalkyl, alkylammoniumalkyl, arylalkyl, carboxyalkyl, carboxyheteroaryl, carboxyheterocycle, carboalkoxyalkyl, carboxyalkylamino, heteroarylalkyl, heterocyclylalkyl, and alkylammoniumalkyl; and
R⁷ and R⁸ are independently selected from the group consisting of hydrogen and alkyl; and
one or more R^{x} are independently selected from the group consisting of H, alkyl, alkenyl, alkynyl, polyalkyl, acyloxy, aryl, arylalkyl, halogen, haloalkyl, cycloalkyl, heterocycle, heteroaryl, polyether, quaternary heterocycle, quaternary heteroaryl, OR¹³, NR¹³R¹⁴, SR¹³, S(O)R¹³, S(O)₂R¹³, SO₃R¹³, S⁺R¹³R¹⁴A⁻, NR¹³OR¹⁴, NR¹³NR¹⁴R¹⁵, NO₂, CO₂R¹³, CN, OM, SO₂OM, SO₂NR¹³R¹⁴, NR¹⁴C(O)R¹³, C(O)NR¹³R¹⁴, NR¹⁴C(O)R¹³, C(O)OM, COR¹³, OR¹⁸, S(O)ₙNR¹⁸, NR¹³R¹⁸, NR¹⁸OR¹⁴, N⁺R⁹R¹¹R¹²A⁻, P⁺R⁹R¹¹R¹²A⁻, amino acid, peptide, polypeptide, and carbohydrate,
wherein alkyl, alkenyl, alkynyl, cycloalkyl, aryl, polyalkyl, heterocycle, acyloxy, arylalkyl, haloalkyl, polyether, quaternary heterocycle, and quaternary heteroaryl can be further substituted with OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₂OM, SO₂NR⁹R¹⁰, PO(OR¹⁶)OR¹⁷, P⁺R⁹R¹¹R¹²A⁻, S⁺R⁹R¹⁰A⁻, or C(O)OM, and
wherein R¹⁸ is selected from the group consisting of acyl, arylalkoxycarbonyl, arylalkyl, heterocycle, heteroaryl, alkyl,
wherein acyl, arylalkoxycarbonyl, arylalkyl, heterocycle, heteroaryl, alkyl, quaternary heterocycle, and quaternary heteroaryl optionally are substituted with one or more substituents selected from the group consisting of OR⁹, NR⁹R¹⁰, N⁺R⁹R¹¹R¹²A⁻, SR⁹, S(O)R⁹, SO₂R⁹, SO₃R⁹, oxo, CO₂R⁹, CN, halogen, CONR⁹R¹⁰, SO₃R⁹, SO₂OM, SO₂NR⁹R¹⁰, PO(OR¹⁶)OR¹⁷, and C(O)OM,
wherein in R^{x}, one or more carbons are optionally replaced by O, NR¹³, N⁺R¹³R¹⁴A⁻, S, SO, SO₂, S⁺R¹³A⁻, PR¹³, P(O)R¹³, P⁺R¹³R¹⁴A⁻, phenylene, amino acid, peptide, polypeptide, carbohydrate, polyether, or polyalkyl,
wherein in said polyalkyl, phenylene, amino acid, peptide, polypeptide, and carbohydrate, one or more carbons are optionally replaced by O, NR⁹, N⁺R⁹R10A⁻, S, SO, SO₂, S⁺R⁹A⁻, PR⁹, P⁺R⁹R¹⁰A⁻, or P(O)R⁹;
wherein quaternary heterocycle and quaternary heteroaryl are optionally substituted with one or more groups selected from the group consisting of alkyl, alkenyl, alkynyl, polyalkyl, polyether, aryl, haloalkyl, cycloalkyl, heterocycle, arylalkyl, halogen, oxo, OR¹³, NR¹³R¹⁴, SR¹³, S(O)R¹³, SO₂R¹³, SO₃R¹³, NR¹³OR¹⁴, NR¹³NR¹⁴R¹⁵, NO₂, CO₂R¹³, CN, OM, SO₂OM, SO₂NR¹³R¹⁴, C(O)NR¹³R¹⁴, C(O)OM, COR¹³, P(O)R¹³R¹⁴, P⁺R¹³R¹⁴R¹⁵A⁻, P(OR¹³)OR¹⁴, S⁺R¹³R¹⁴A⁻, and N⁺R⁹R¹¹R¹²A⁻, or a pharmaceutically acceptable salt thereof.

In some embodiments, the ASBT inhibitor is a compound of formula (IV):

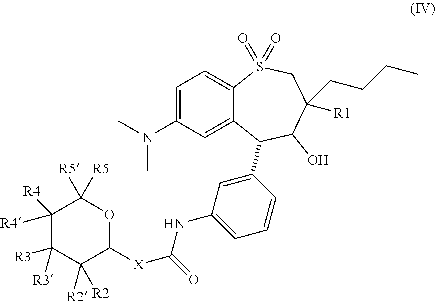

wherein
X is O, NH, CH₂ or a bond;
R¹ is C₁₋₆alkyl;
R², R^{2′}, R³, R^{3′}, R⁴, R^{4′}, R⁵ and R^{5′} are each independently selected from the group consisting of H, Cl, Br, I, OH, —(CH₂)—OH, CF₃, NO₂, N₃, CN, S(O)ₚ—R⁶, O—S(O)ₚ—R⁶, C₁₋₆alkylene-S(O)ₚ-R⁶, C₁₋₆alkylene-O-S(O)ₚ-R⁶, COOH, COOC₁₋₆alkyl, CONH₂, CONHC₁₋₆alkyl, CON(C₁₋₆alkyl)₂, C₁₋₆alkyl, C₂₋₆alkenyl, C2-6alkynyl and O—C₁₋₆alkyl, wherein one or more of the alkyl hydrogens may be replaced by fluorine; and phenyl, —(CH₂)-phenyl, —(CH₂)ₙ-phenyl, O-phenyl, O—(CH₂)ₘ-phenyl, —(CH₂)—O—(CH₂)ₘ-phenyl, wherein the phenyl ring may be substituted one to three times by F, Cl, Br, I, OH, CF₃, NO₂, CN, OCF₃, O—C₁₋₆alkyl, C₁₋₆alkyl, NH₂, NHC₁₋₆alkyl, N(C₁₋₆alkyl)₂, SO₂—CH₃—, COOH, COOC₁₋₆alkyl, or CONH₂;
wherein always at least one of R², R_{2′}, R₃, R_{3′}, R₄, R_{4′}, R₅, R_{5′} is —O—(CH₂)ₘ-phenyl or —(CH₂)—O—(CH₂)ₘ-phenyl, wherein the phenyl ring may be substituted one to 3 times by F, Cl, Br, I, OH, CF₃, NO₂, CN, OCF₃, O—C₁₋₆alkyl, C₁₋₆alkyl, NH₂, NHC₁₋₆alkyl, N(C₁₋₆alkyl)₂, SO₂—CH₃, COOH, COOC₁₋₆alkyl, CONH₂;
R⁶ is selected from the group consisting of H, OH, C₁₋₆alkyl, NH₂, NHC₁₋₆alkyl and N(C₁₋₆alkyl)₂;
n is an integer 2, 3, 4, 5 or 6;
m is an integer 1, 2, 3, 4, 5 or 6; and
p is an integer 0, 1 or 2;

or a pharmaceutically acceptable salt thereof.

In some embodiments, the ASBT inhibitor is a compound of formula (V):

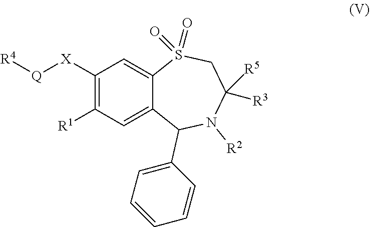

wherein
R¹ is selected from the group consisting of H, Cl, Br, N(CH₃)₂ and methoxy;
R² is H or OH;
each R³ is independently C₁₋₆alkyl;
X is CH₂, C(O) or CH═CH;
Q is C₀₋₆alkyl;
R⁴ is selected from the group consisting of OH, SO₃H, CO₂H, PO₃H₂, CONR⁵R⁵, NR⁵R⁵ and NHC(O)CH₂NR⁵R⁵;
each R⁵ is independently selected from the group consisting of H, OH, C₁₋₆alkyl, C₀₋₆alkylCO₂H, C₀₋₆alkylSO₃H, C₀₋₆alkylPO₃H₂, C(O)C₀₋₆alkylCO₂H, C(O)C₀₋₆alkylSO₃H, C(O)C₀₋₆alkylPO₃H₂ and CH(R⁶)C₀₋₆alkylCO₂H; and
R⁶ is selected from the group consisting of C₀₋₆alkylCO₂H, C₀₋₆alkylOH, C₀₋₆alkylSO₃H and C₀₋₆alkylPO₃H₂;

or a pharmaceutically acceptable salt thereof.

In some embodiments, the ASBT inhibitor is a compound of formula (VI):

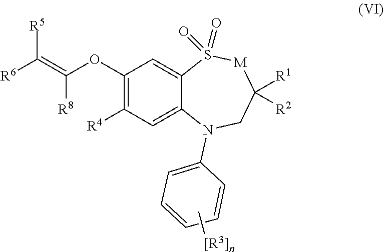

wherein
M is selected from —CH₂— and —NR⁷—;
R¹ and R² are each independently C₁₋₄alkyl;
R³ is selected from the group consisting of hydrogen, halogen, hydroxy, C₁₋₄alkyl, C₁₋₄haloalkyl, C₁₋₄alkoxy, cyano, nitro, amino, N-(C₁₋₄alkyl)amino, N,N-di(C₁₋₄alkyl)amino, N-(aryl-C₁₋₄alkyl)amino, C₁₋₆alkylcarbonylamino, C₃₋₆cycloalkylcarbonylamino, N-(C₁₋₄alkyl)aminocarbonyl, N,N-di(C₁₋₄alkyl)aminocarbonyl, C₁₋₄alkyloxycarbonylamino, C₃₋₆cycloalkyloxycarbonylamino, C₁₋₄alkylsulfonamido and C₃₋₆cycloalkylsulfonamido;
n is an integer 1, 2 or 3;
R⁴ is selected from the group consisting of hydrogen, halogen, cyano, C₁₋₄alkyl, C₃₋₆cycloalkyl, C₁₋₄alkoxy, C₃₋₆cycloalkyloxy, C₁₋₄alkylthio, C₃₋₆cycloalkylthio, amino, N-(C₁₋₄alkyl)amino and N,N-di(C₁₋₄alkyl)amino;
one of R⁵ and R⁶ is carboxy, and the other of R⁵ and R⁶ is selected from the group consisting of hydrogen, fluoro, C₁₋₄alkyl and C₁₋₄haloalkyl;
R⁷ is selected from the group consisting of hydrogen and C₁₋₄alkyl; and
R⁸ is selected from the group consisting of hydrogen and C₁₋₄alkyl;

or a pharmaceutically acceptable salt thereof.

In some embodiments, the ASBT inhibitor is a compound of formula (VII):

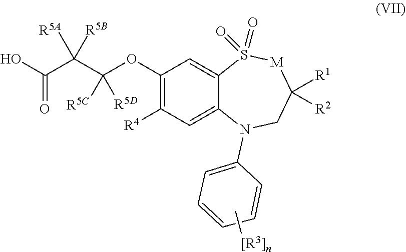

wherein
M is —CH₂— or —NR⁶—;
R¹ and R² are each independently C₁₋₄alkyl;
R³ is independently selected from the group consisting of hydrogen, halogen, hydroxy, C₁₋₄alkyl, C₁₋₄haloalkyl, C₁₋₄alkoxy, C₁₋₄haloalkoxy, cyano, nitro, amino, N-(C₁₋₄alkyl)amino, N,N-di(C₁₋₄alkyl)amino and N-(aryl-C₁₋₄alkyl)amino;
n is an integer 1, 2 or 3;
R⁴ is selected from the group consisting of hydrogen, halogen, hydroxy, cyano, C₁₋₄alkyl, C₃₋₆cycloalkyl, C₁₋₄alkoxy, C₃₋₆cycloalkyloxy, C₁₋₄alkylthio, C₃₋₆cycloalkylthio, amino, N-(C₁₋₄alkyl)amino and N,N-di(C₁₋₄alkyl)amino; and
R^{5A}, R^{5B}, R^{5C} and R^{5D} are each independently selected from the group consisting of hydrogen, halogen, hydroxy, amino, C₁₋₄alkyl and C₁₋₄alkoxy; and
R⁶ is selected from the group consisting of hydrogen and C₁₋₄alkyl; or a pharmaceutically acceptable salt thereof.

In some embodiments, the ASBT inhibitor is a compound of formula (VIII):

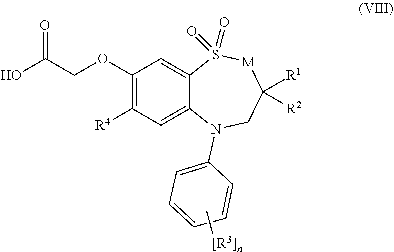

wherein
M is —CH₂— or —NH—;
R¹ and R² are each independently C₁₋₄alkyl;
R³ is independently selected from the group consisting of hydrogen, halogen, hydroxy, C₁₋₄alkyl, C₁₋₄haloalkyl, C₁₋₄alkoxy, C₁₋₄haloalkoxy, cyano, nitro, amino, N-(C₁₋₄alkyl)amino, N,N-di(C₁₋₄alkyl)amino, and N-(aryl-C₁₋₄alkyl)amino;
n is an integer 1, 2 or 3; and
R⁴ is selected from the group consisting of hydrogen, halogen, hydroxy, cyano, C₁₋₄alkyl, C₃₋₆cycloalkyl, C₁₋₄alkoxy, C₃₋₆cycloalkyloxy, C₁₋₄alkylthio, C₃₋₆cycloalkylthio, amino, N-(C₁₋₄alkyl)amino and N,N-di(C₁₋₄alkyl)amino;

or a pharmaceutically acceptable salt thereof.

In some embodiments, the ASBT inhibitor is a compound of formula (IX):

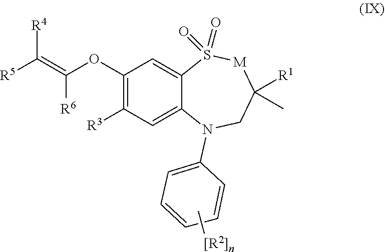

wherein
M is selected from —CH₂— and —NR⁶—;
R¹ is C₁₋₄alkyl;
R² is independently selected from the group consisting of hydrogen, halogen, hydroxy, C₁₋₄alkyl, C₁₋₄haloalkyl, C₁₋₄alkoxy, cyano, nitro, amino, N-(C₁₋₄alkyl)amino, N,N-di(C₁₋₄alkyl)amino, N-(aryl-C₁₋₄alkyl)amino, C₁₋₆alkylcarbonylamino, C₃₋₆cycloalkylcarbonylamino, N-(C₁₋₄alkyl)aminocarbonyl, N,N-di(C₁₋₄alkyl)aminocarbonyl, C₁₋₄alkyloxycarbonylamino, C₃₋₆cycloalkyloxycarbonylamino, C₁₋₄alkylsulfonamido and C₃₋₆cycloalkylsulfonamido;
n is an integer 1, 2 or 3;
R³ is selected from the group consisting of hydrogen, halogen, cyano, C₁₋₄alkyl, C₃₋₆cycloalkyl, C₁₋₄ alkoxy, C₃₋₆cycloalkyloxy, C₁₋₄alkylthio, C₃₋₆cycloalkylthio, amino, N-(C₁₋₄alkyl)amino and N,N-di(C₁₋₄alkyl)amino;
one of R⁴ and R⁵ is carboxyl, and the other of R⁴ and R⁵ is selected from the group consisting of hydrogen, fluoro, C₁₋₄alkyl and C₁₋₄haloalkyl;
R⁶ is selected from the group consisting of hydrogen and C₁₋₄alkyl; and
R⁷ is selected from the group consisting of hydrogen and C₁₋₄alkyl;

or a pharmaceutically acceptable salt thereof.

In some embodiments, the ASBT inhibitor is a compound of formula (X):

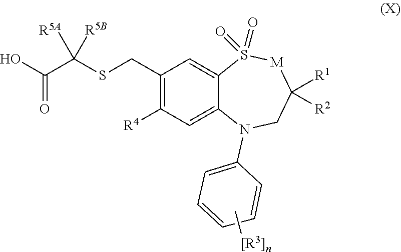

wherein
M is —CH₂— or —NR⁶—;
R¹ is C₁₋₄alkyl;
R² is selected from the group consisting of hydrogen and C₁₋₄alkyl;
R³ is independently selected from the group consisting of hydrogen, halogen, hydroxy, C₁₋₄alkyl, C₁₋₄haloalkyl, C₁₋₄alkoxy, C₁₋₄haloalkoxy, cyano, nitro, amino, N-(C₁₋₄alkyl)amino and N,N-di(C₁₋₄alkyl)amino;
n is an integer 1, 2 or 3;
R⁴ is selected from the group consisting of hydrogen, halogen, cyano, C₁₋₄alkyl, C₃₋₆cycloalkyl, C₁₋₄alkoxy, C₃₋₆cycloalkyloxy, C₁₋₄alkylthio, C₃₋₆cycloalkylthio, amino, N-(C₁₋₄alkyl)amino and N,N-di(C₁₋₄alkyl)amino;
R^{5A} and R^{5B} are each independently selected from the group consisting of hydrogen, halogen, hydroxy, C₁₋₄alkyl and C₁₋₄alkoxy; or R^{5A} and R^{5B}, together with the carbon atom to which they are attached, form a 3- to 5-membered saturated carbocyclic ring; and
R⁶ is selected from the group consisting of hydrogen and C₁₋₄alkyl;

or a pharmaceutically acceptable salt thereof.

In some embodiments, the ASBT inhibitor is a compound selected from the group consisting of:

1,1-dioxo-3,3-dibutyl-5-phenyl-7-methylthio-8-(N-{(R)-1′-phenyl-1′-[N′-(carboxymethyl)-carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N-((S)-1-carboxypropyl)carbamoyl]-4-hydroxybenzyl}carbamoylmethoxy)-2,3,4,5-tetrahydro-1,2,5-benzothiadiazepine;
1-{[4-({4-[(4R,5R)-3,3-dibutyl-7-(dimethylamino)-4-hydroxy-1,1-dioxo-2,3,4,5-tetrahydro-1H-1λ⁶-benzothiepin-5-yl]phenoxy}methyl)phenyl]methyl}-1,4-diazabicyclo[2.2.2]octan-1-ium chloride;
N-(3-O-benzyl-6-O-sulfo-β-D-glucopyranosyl)-N′-{3-[(3S,4R,5R)-3-butyl-7-(dimethylamino)-3-ethyl-4-hydroxy-1,1-dioxo-2,3,4,5-tetrahydro-1H-1λ⁶-benzothiepin-5-yl]phenyl}urea;
3-({[(3R,5R)-3-butyl-3-ethyl-7-methoxy-1,1-dioxo-5-phenyl-2,3,4,5-tetrahydro-1H-1λ⁶,4-benzothiazepin-8-yl]methyl}amino)pentanedioic acid;
(Z)-3-((3,3-dibutyl-7-(methylthio)-1,1-dioxido-5-phenyl-2,3,4,5-tetrahydro-1,5-benzothiazepin-8-yl)oxy)-2-fluoroacrylic acid;
(Z)-3-((3-butyl-3-ethyl-7-(methylthio)-1,1-dioxido-5-phenyl-2,3,4,5-tetrahydro-1,5-benzothiazepin-8-yl)oxy)-2-fluoroacrylic acid;
3-((7-bromo-3-butyl-3-ethyl-1,1-dioxido-5-phenyl-2,3,4,5-tetrahydro-1,5-benzothiazepin-8-yl)oxy)propanoic acid;
3-((3,3-dibutyl-7-(methylthio)-1,1-dioxido-5-phenyl-2,3,4,5-tetrahydro-1,5-benzothiazepin-8-yl)oxy)-2-hydroxypropanoic acid;
3-((3-butyl-3-ethyl-7-(methylthio)-1,1-dioxido-5-phenyl-2,3,4,5-tetrahydro-1,5-benzothiazepin-8-yl)oxy)propanoic acid;
3-((3,3-dibutyl-7-(methylthio)-1,1-dioxido-5-phenyl-2,3,4,5-tetrahydro-1,2,5-benzothiadiazepin-8-yl)oxy)propanoic acid;
3-((3-butyl-3-ethyl-7-(methylthio)-1,1-dioxido-5-phenyl-2,3,4,5-tetrahydro-1,2,5-benzothiadiazepin-8-yl)oxy)propanoic acid;
2-((3-butyl-7-(dimethylamino)-3-ethyl-1,1-dioxido-5-phenyl-2,3,4,5-tetrahydro-1,5-benzothiazepin-8-yl)oxy)acetic acid;
2-((3-butyl-3-ethyl-7-(methylthio)-1,1-dioxido-5-phenyl-2,3,4,5-tetrahydrobenzo-1,2,5-thiadiazepin-8-yl)oxy)acetic acid; and
(E)-3-((3-butyl-3-ethyl-5-(4-fluorophenyl)-7-(methylthio)-1,1-dioxido-2,3,4,5-tetrahydro-1,5-benzothiazepin-8-yl)oxy)acrylic acid;

or a pharmaceutically acceptable salt thereof.

In some embodiments, the ASBT inhibitor is (Z)-3-((3-butyl-3-ethyl-7-(methylthio)-1,1-dioxido-5-phenyl-2,3,4,5-tetrahydro-1,5-benzothiazepin-8-yl)oxy)-2-fluoroacrylic acid:

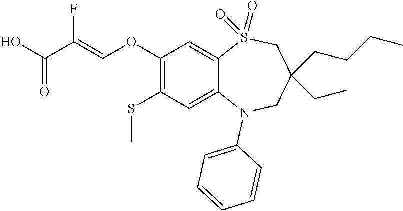 [SMILES: CCCCC1(CC)CN(c2ccccc2)c2cc(SC)c(O/C=C(\F)C(=O)O)cc2S(=O)(=O)C1]

or a pharmaceutically acceptable salt thereof, also referred to herein as “Compound 1”. In some embodiments, the ASBT inhibitor is (S)-(Z)-3-((3-butyl-3-ethyl-7-(methylthio)-1,1-dioxido-5-phenyl-2,3,4,5-tetrahydro-1,5-benzothiazepin-8-yl)oxy)-2-fluoroacrylic acid, or a pharmaceutically acceptable salt thereof. In some embodiments, Compound 1 is (S)-(Z)-3-((3-butyl-3-ethyl-7-(methylthio)-1,1-dioxido-5-phenyl-2,3,4,5-tetrahydro-1,5-benzothiazepin-8-yl)oxy)-2-fluoroacrylic acid. In some embodiments, the ASBT inhibitor is (R)-(Z)-3-((3-butyl-3-ethyl-7-(methylthio)-1,1-dioxido-5-phenyl-2,3,4,5-tetrahydro-1,5-benzothiazepin-8-yl)oxy)-2-fluoroacrylic acid, or a pharmaceutically acceptable salt thereof. In some embodiments, Compound 1 is (R)-(Z)-3-((3-butyl-3-ethyl-7-(methylthio)-1,1-dioxido-5-phenyl-2,3,4,5-tetrahydro-1,5-benzothiazepin-8-yl)oxy)-2-fluoroacrylic acid. Compound 1 can be prepared as described in WO 2019/234077.

In some embodiments, the ASBT inhibitor is 3-((3,3-dibutyl-7-(methylthio)-1,1-dioxido-5-phenyl-2,3,4,5-tetrahydro-1,2,5-benzothiadiazepin-8-yl)oxy)propanoic acid:

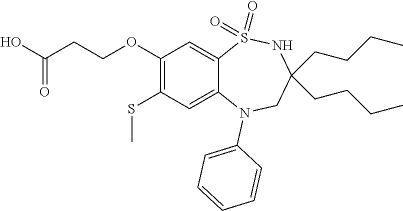 [SMILES: CCCCC1(CCCC)CN(c2ccccc2)c2cc(SC)c(OCCC(=O)O)cc2S(=O)(=O)N1]

or a pharmaceutically acceptable salt thereof, also referred to herein as “Compound 2”. Compound 2 can be prepared as described in WO 2020/161217.

In some embodiments, the ASBT inhibitor is 2-(((3-butyl-3-methyl-7-(methylthio)-1,1-dioxido-5-phenyl-2,3,4,5-tetrahydro-1,5-benzothiazepin-8-yl)methyl)thio)acetic acid:

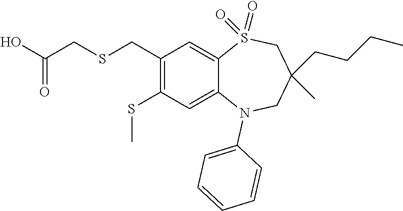 [SMILES: CCCCC1(C)CN(c2ccccc2)c2cc(SC)c(CSCC(=O)O)cc2S(=O)(=O)C1]

or a pharmaceutically acceptable salt thereof, also referred to herein as “Compound 3”. In some embodiments, the ASBT inhibitor is (S)-2-(((3-butyl-3-methyl-7-(methylthio)-1,1-dioxido-5-phenyl-2,3,4,5-tetrahydro-1,5-benzothiazepin-8-yl)methyl)thio)acetic acid, or a pharmaceutically acceptable salt thereof. In some embodiments, Compound 3 is (S)-2-(((3-butyl-3-methyl-7-(methylthio)-1,1-dioxido-5-phenyl-2,3,4,5-tetrahydro-1,5-benzothiazepin-8-yl)methyl)thio)acetic acid. In some embodiments, the ASBT inhibitor is (R)-2-(((3-butyl-3-methyl-7-(methylthio)-1,1-dioxido-5-phenyl-2,3,4,5-tetrahydro-1,5-benzothiazepin-8-yl)methyl)thio)acetic acid, or a pharmaceutically acceptable salt thereof. In some embodiments, Compound 3 is (R)-2-(((3-butyl-3-methyl-7-(methylthio)-1,1-dioxido-5-phenyl-2,3,4,5-tetrahydro-1,5-benzothiazepin-8-yl)methyl)thio)acetic acid. Compound 2 can be prepared as described in PCT/EP2023/068476.

In some embodiments, the ASBT inhibitor is 2-(((3-butyl-7-methoxy-3-methyl-1,1-dioxido-5-phenyl-2,3,4,5-tetrahydro-1,5-benzothiazepin-8-yl)methyl)thio)acetic acid:

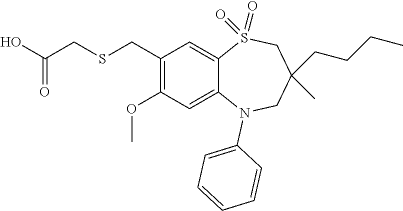 [SMILES: CCCCC1(C)CN(c2ccccc2)c2cc(OC)c(CSCC(=O)O)cc2S(=O)(=O)C1]

or a pharmaceutically acceptable salt thereof, also referred to herein as “Compound 4”. In some embodiments, the ASBTI inhibitor is (S)-2-(((3-butyl-7-methoxy-3-methyl-1,1-dioxido-5-phenyl-2,3,4,5-tetrahydro-1,5-benzothiazepin-8-yl)methyl)thio)acetic acid, or a pharmaceutically acceptable salt thereof. In some embodiments, Compound 4 is (S)-2-(((3-butyl-7-methoxy-3-methyl-1,1-dioxido-5-phenyl-2,3,4,5-tetrahydro-1,5-benzothiazepin-8-yl)methyl)thio)acetic acid. In some embodiments, the ASBTI inhibitor is (R)-2-(((3-butyl-7-methoxy-3-methyl-1,1-dioxido-5-phenyl-2,3,4,5-tetrahydro-1,5-benzothiazepin-8-yl)methyl)thio)acetic acid, or a pharmaceutically acceptable salt thereof. In some embodiments, Compound 4 is (R)-2-(((3-butyl-7-methoxy-3-methyl-1,1-dioxido-5-phenyl-2,3,4,5-tetrahydro-1,5-benzothiazepin-8-yl)methyl)thio)acetic acid. Compound 4 can be prepared as described in PCT/EP2023/068476.

In some embodiments, the ASBT inhibitor is 2-((3-butyl-3-ethyl-5-(4-fluorophenyl)-7-(methylthio)-1,1-dioxido-2,3,4,5-tetrahydro-1,5-benzothiazepin-8-yl)oxy)acetic acid:

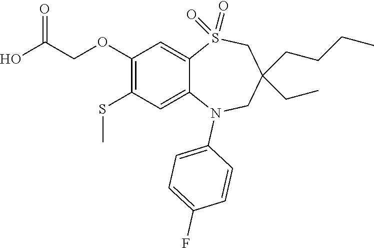 [SMILES: CCCCC1(CC)CN(c2ccc(F)cc2)c2cc(SC)c(OCC(=O)O)cc2S(=O)(=O)C1]

or a pharmaceutically acceptable salt thereof, also referred to herein as “Compound 5”. In some embodiments, the ASBTI inhibitor is (S)-2-((3-butyl-3-ethyl-5-(4-fluorophenyl)-7-(methylthio)-1,1-dioxido-2,3,4,5-tetrahydro-1,5-benzothiazepin-8-yl)oxy)acetic acid, or a pharmaceutically acceptable salt thereof. In some embodiments, Compound 5 is (S)-2-((3-butyl-3-ethyl-5-(4-fluorophenyl)-7-(methylthio)-1,1-dioxido-2,3,4,5-tetrahydro-1,5-benzothiazepin-8-yl)oxy)acetic acid. In some embodiments, the ASBTI inhibitor is (R)-2-(3-butyl-3-ethyl-5-(4-fluorophenyl)-7-(methylthio)-1,1-dioxido-2,3,4,5-tetrahydro-1,5-benzothiazepin-8-yl)oxy)acetic acid, or a pharmaceutically acceptable salt thereof. In some embodiments, Compound 5 is (R)-2-(3-butyl-3-ethyl-5-(4-fluorophenyl)-7-(methylthio)-1,1-dioxido-2,3,4,5-tetrahydro-1,5-benzothiazepin-8-yl)oxy)acetic acid. Compound 5 can be prepared as described in WO 2021/110887.

In some embodiments, the ASBT inhibitor is a compound selected from:

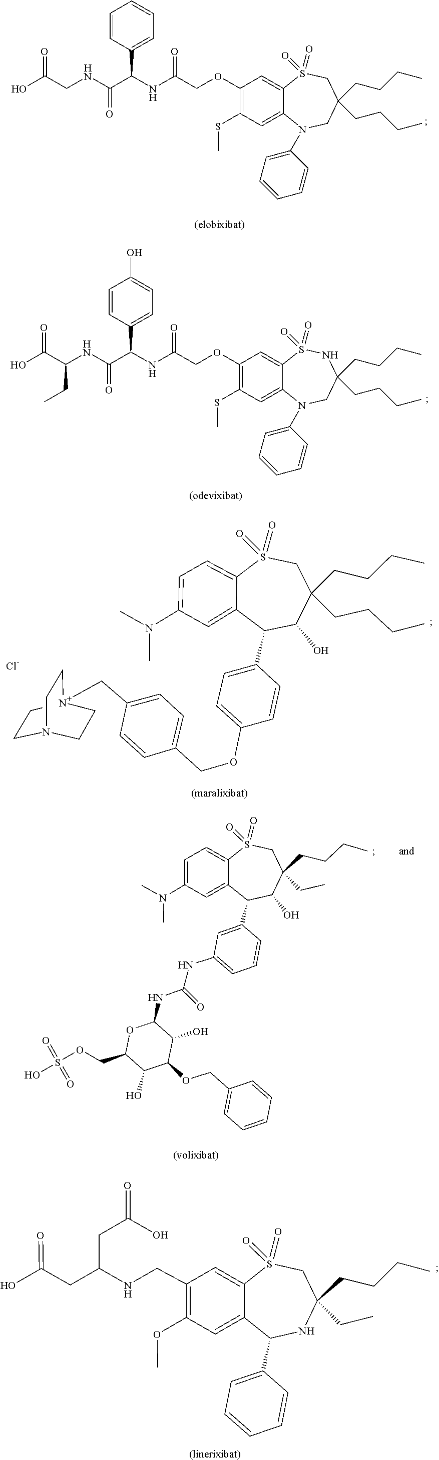

or a pharmaceutically acceptable salt thereof.

In some embodiments, the ASBT inhibitor is elobixibat, or a pharmaceutically acceptable salt thereof. In some embodiments, the ASBT inhibitor is odevixibat, or a pharmaceutically acceptable salt thereof. In some embodiments, the ASBT inhibitor is maralixibat, or a pharmaceutically acceptable salt thereof. In some embodiments, the ASBT inhibitor is volixibat, or a pharmaceutically acceptable salt thereof. In some embodiments, the ASBT inhibitor is linerixibat, or a pharmaceutically acceptable salt thereof. In some embodiments, the ASBT inhibitor comprises a combination of two or more of elobixibat, odevixibat, maralixibat, volixibat, and linerixibat, or a pharmaceutically acceptable salt thereof.

As used herein, the term “halo” refers to fluoro, chloro, bromo and iodo.

As used herein, the term “C₁₋₆alkyl” refers to a straight or branched alkyl group having from 1 to 6 carbon atoms, and the term “C₁₋₄alkyl” refers to a straight or branched alkyl group having from 1 to 4 carbon atoms. Examples of C₁₋₄alkyl include methyl, ethyl, n-propyl, isopropyl, n-butyl, isobutyl, sec-butyl and tert-butyl.

As used herein, the term “C₁₋₄haloalkyl” refers to a straight or branched C₁₋₄alkyl group, as defined herein, wherein one or more hydrogen atoms have been replaced with halogen. Examples of C₁₋₄haloalkyl include chloromethyl, fluoroethyl and trifluoromethyl.

As used herein, the terms “C₁₋₄alkoxy” and “C₁₋₄alkylthio” refer to a straight or branched C₁₋₄alkyl group attached to the remainder of the molecule through an oxygen or sulphur atom, respectively.

As used herein, the term “C₃₋₆cycloalkyl” refers to a monocyclic saturated hydrocarbon ring having from 3 to 6 carbon atoms. Examples of C₃₋₆cycloalkyl include cyclopropyl, cyclobutyl, cyclopentyl and cyclohexyl.

The term “amino” refers to an −NH₂ group. As used herein, the terms “N-(C₁₋₄alkyl)amino” and “N,N-di(C₁₋₄alkyl)amino” refer to an amino group wherein one or both hydrogen atom(s), respectively, are replaced with a straight or branched C₁₋₄alkyl group. Examples of N-(C₁₋₄alkyl)amino include methylamino, ethylamino and tert-butylamino, and examples of N, N-di-(C₁₋₄alkyl)amino include dimethylamino and diethylamino.

The term “aryl” denotes an aromatic monocyclic ring composed of 6 carbon atoms or an aromatic bicyclic ring system composed of 10 carbon atoms. Examples of aryl include phenyl, naphthyl and azulenyl.

As used herein, the term “N-(aryl-C₁₋₄alkyl)amino” refers to an amino group wherein a hydrogen atom is replaced with an aryl-C₁₋₄alkyl group. Examples of N-(aryl-C₁₋₄alkyl)amino include benzylamino and phenylethylamino. The term “C₁₋₆alkylcarbonylamino” refers to an amino group wherein a hydrogen atom is replaced with a C₁₋₆alkylcarbonyl group. Examples of C₁₋₆alkanoylamino include acetylamino and tert-butylcarbonylamino. The term “C₁₋₄alkyloxycarbonylamino” refers to an amino group wherein a hydrogen atom is replaced with a C₁₋₄alkyloxycarbonyl group. An example of C₁₋₄alkyloxycarbonylamino is tert-butoxycarbonylamino. The terms “C₁₋₄alkylsulfonamido” and “C₃₋₆cycloalkylsulfonamido” refer to an amino group wherein a hydrogen atom is replaced with a C₁₋₄alkylsulfonyl or a C₃₋₆cycloalkylsulfonyl group, respectively.

Some ASBT inhibitors, or pharmaceutically acceptable salts thereof, may have chiral centres and/or geometric isomeric centres (E- and Z-isomers). It is to be understood that the invention encompasses all such optical isomers, diastereoisomers and geometric isomers that possess ASBT inhibitory activity. The invention also encompasses any and all tautomeric forms that possess ASBT inhibitory activity. Certain ASBT inhibitors, or pharmaceutically acceptable salts thereof, may exist in unsolvated as well as solvated forms, such as, for example, hydrated forms. It is to be understood that the invention encompasses all such solvated forms that possess ASBT inhibitory activity.

As used herein, the term “pharmaceutically acceptable” refers to those compounds, materials, compositions and/or dosage forms that are suitable for human pharmaceutical use and that are generally safe, non-toxic and neither biologically nor otherwise undesirable.

A suitable pharmaceutically acceptable salt of an ASBT inhibitor is, for example, a base-addition salt of such a compound which is sufficiently acidic, such as an alkali metal salt (e.g., a sodium or potassium salt), an alkaline earth metal salt (e.g., a calcium or magnesium salt), an ammonium salt, or a salt with an organic base which affords a physiologically acceptable cation, for example a salt with methylamine, dimethylamine, trimethylamine, piperidine, morpholine or tris-(2-hydroxyethyl)amine.

### Administration of the ASBT Inhibitor

In some embodiments, following administration of the ASBT inhibitor, or a pharmaceutically acceptable salt thereof, the subject exhibits a reduction in serum bile acid concentration of at least 50% relative to baseline (e.g., at least 55%; at least 60; at least 65%; at least 70%; at least 75%; at least 80%; at least 85%; at least 90%; or at least 95%). In some embodiments, the subject exhibits a reduction in serum bile acid concentration of at least 60%, at least 70%, at least 80%, or at least 90% relative to baseline.

In some embodiments, following administration of the ASBT inhibitor, or a pharmaceutically acceptable salt thereof, the subject exhibits a reduction in serum bile acid concentration of about 50% to about 100% relative to baseline (e.g., about 50% to about 60%; about 50% to about 70%; about 50% to about 80%; about 50% to about 90%; about 60% to about 70%; about 60% to about 80%; about 60% to about 90%; about 60% to about 100%; about 70% to about 80%; about 70% to about 90%; about 70% to about 100%; about 80% to about 90%; about 80% to about 100%; or about 90% to about 100%). In some embodiments, the subject exhibits a reduction in serum bile acid concentration of about 50%, about 60%, about 70%, about 80%, or about 90% relative to baseline.

In some embodiments, the serum bile acid concentration is normalized following administration of the ASBT inhibitor, or a pharmaceutically acceptable salt thereof. In some embodiments, the serum bile acid concentration is normalized following administration of the ASBT inhibitor, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc.

In some embodiments, the serum bile acid concentration is normalized following administration of the ASBT inhibitor, or a pharmaceutically acceptable salt thereof, for about 1 week to about 72 weeks (e.g., about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 16 weeks, about 4 weeks to about 24 weeks, about 4 weeks to about 40 weeks, about 4 weeks to about 52 weeks, about 4 weeks to about 72 weeks, about 8 weeks to about 16 weeks, about 8 weeks to about 24 weeks, about 8 weeks to about 36 weeks, about 8 weeks to about 48 weeks, about 8 weeks to about 60 weeks, about 12 weeks to about 20 weeks, about 12 weeks to about 28 weeks, about 12 weeks to about 40 weeks, about 12 weeks to about 52 weeks, about 12 weeks to about 72 weeks, about 16 weeks to about 24 weeks, about 16 weeks to about 36 weeks, about 16 weeks to about 48 weeks, about 16 weeks to about 60 weeks, about 20 weeks to about 28 weeks, about 20 weeks to about 40 weeks, about 20 weeks to about 52 weeks, about 20 weeks to about 72 weeks, about 24 weeks to about 36 weeks, about 24 weeks to about 48 weeks, about 24 weeks to about 60 weeks, about 28 weeks to about 40 weeks, about 28 weeks to about 52 weeks, about 28 weeks to about 72 weeks, about 36 weeks to about 48 weeks, about 36 weeks to about 60 weeks, about 40 weeks to about 44 weeks, about 40 weeks to about 52 weeks, about 40 weeks to about 72 weeks, about 44 weeks to about 52 weeks, about 44 weeks to about 72 weeks, about 48 weeks to about 60 weeks, about 52 weeks to about 72 weeks, or about 60 weeks to about 72 weeks.

In some embodiments, following administration of the ASBT inhibitor, or a pharmaceutically acceptable salt thereof, the subject exhibits an increase in urinary bile acids of at least 50% relative to baseline (e.g., at least 55%; at least 60; at least 65%; at least 70%; at least 75%; at least 80%; at least 85%; at least 90%; or at least 95%). In some embodiments, the subject exhibits an increase in urinary bile acids of at least 60%, at least 70%, at least 80%, or at least 90% relative to baseline.

In some embodiments, following administration of the ASBT inhibitor, or a pharmaceutically acceptable salt thereof, the subject exhibits an increase in urinary bile acids of about 50% to about 100% relative to baseline (e.g., about 50% to about 60%; about 50% to about 70%; about 50% to about 80%; about 50% to about 90%; about 60% to about 70%; about 60% to about 80%; about 60% to about 90%; about 60% to about 100%; about 70% to about 80%; about 70% to about 90%; about 70% to about 100%; about 80% to about 90%; about 80% to about 100%; or about 90% to about 100%). In some embodiments, the subject exhibits an increase in urinary bile acids of about 50%, about 60%, about 70%, about 80%, or about 90% relative to baseline.

In some embodiments, the presence of a disease recited herein, such as cholemic nephropathy, is determined by one or more biomarkers indicative of one or more of bile duct obstruction, cholestasis, inflammation, liver fibrosis, liver cirrhosis and/or scoring systems thereof. In some embodiments, the severity of a disease recited herein, such as cholemic nephropathy, is determined by one or more biomarkers indicative of one or more of bile duct obstruction, cholestasis, inflammation, liver fibrosis, liver cirrhosis and/or scoring systems thereof. In some embodiments, the result of the treatment of a disease recited herein, such as cholemic nephropathy, is determined by one or more biomarkers indicative of one or more of bile duct obstruction, cholestasis, inflammation, liver fibrosis, liver cirrhosis and/or scoring systems thereof. Non-limiting examples of biomarkers indicative of one or more of bile duct obstruction, cholestasis, inflammation, liver fibrosis, liver cirrhosis and/or scoring systems thereof include levels of alanine transaminase (ALT), aspartate transaminase (AST), alkaline phosphatase (ALP), gamma-glutamyl transferase (GGT), serum bilirubin, prothrombin time (PT), the international normalized ratio (INR), total protein and albumin (see, e.g., Lala et al., “Liver Function Tests.” StatPearls, StatPearls Publishing, 5 October 2022 (PMID: 29494096), which is incorporated by reference herein in its entirety). In some embodiments, the subject exhibits an improvement in liver parameters (biomarkers) following administration of the ASBT inhibitor, or a pharmaceutically acceptable salt thereof. In some embodiments, the level of aspartate aminotransferase (AST) does not increase. In some embodiments, the level of aspartate aminotransferase (AST) decreases. In some embodiments, the level of alanine aminotransferase (ALT) does not increase. In some embodiments, the level of alanine aminotransferase (ALT) decreases. In some embodiments, the “level” of an enzyme refers to the concentration of the enzyme, e.g., within blood. For example, the level of AST or ALT can be expressed as Units/L.

In some embodiments, serum total bilirubin levels are decreased following administration of the ASBT inhibitor, or a pharmaceutically acceptable salt thereof. In some embodiments, total bilirubin levels are decreased by about 0.5 mg/dL to about 5.0 mg/dl, about 1 mg/dl to about 5.0 mg/dL, about 1.5 mg/dl to about 5.0 mg/dl, or about 2.0 mg/dl to about 5.0 mg/dl from baseline following administration of the ASBT inhibitor, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, total bilirubin can be reduced at least 70% (e.g., approximately 99%) following administration of the ASBT inhibitor, or a pharmaceutically acceptable salt thereof, for at least 24 weeks.

In some embodiments, total bilirubin levels are decreased by about 0.5 mg/dl to about 5.0 mg/dl, about 0.5 mg/dl to about 4.0 mg/dl, about 0.5 mg/dl to about 3.0 mg/dl, about 0.5 mg/dl to about 2.0 mg/dl, about 0.5 mg/dl to about 1.5 mg/dl, about 1.0 mg/dl to about 5.0 mg/dl, about 1.0 mg/dl to about 4.0 mg/dL, about 1.0 mg/dl to about 3.0 mg/dl, about 1.0 mg/dl to about 2.0 mg/dl, about 1.0 mg/dl to about 1.5 mg/dl, about 1.5 mg/dl to about 5.0 mg/dl, about 1.5 mg/dl to about 4.0 mg/dl, about 1.5 mg/dL to about 3.0 mg/dl, about 1.5 mg/dl to about 2.0 mg/dL, about 2.0 mg/dl to about 5.0 mg/dl, about 2.0 mg/dl to about 4.0 mg/dl, about 2.0 mg/dL to about 3.0 mg/dl, about 3.0 mg/dl to about 5.0 mg/dl, about 3.0 mg/dl to about 4.0 mg/dl, or 4.0 mg/dL to about 5.0 mg/dl from baseline following administration of the ASBT inhibitor, or a pharmaceutically acceptable salt thereof. For example, total bilirubin can be reduced by about 50% to about 100% (e.g., about 50% to about 60%; about 50% to about 70%; about 50% to about 80%; about 50% to about 90%; about 60% to about 70%; about 60% to about 80%; about 60% to about 90%; about 60% to about 100%; about 70% to about 80%; about 70% to about 90%; about 70% to about 100%; about 80% to about 90%; about 80% to about 100%; or about 90% to about 100%) following administration of the ASBT inhibitor, or a pharmaceutically acceptable salt thereof. In some embodiments, serum total bilirubin levels are decreased by about 50%, about 60%, about 70%, about 80%, or about 90% relative to baseline following administration of the ASBT inhibitor, or a pharmaceutically acceptable salt thereof. In some embodiments, serum total bilirubin levels are decreased following administration of the ASBT inhibitor, or a pharmaceutically acceptable salt thereof, for about 1 week to about 72 weeks (e.g., about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 16 weeks, about 4 weeks to about 24 weeks, about 4 weeks to about 40 weeks, about 4 weeks to about 52 weeks, about 4 weeks to about 72 weeks, about 8 weeks to about 16 weeks, about 8 weeks to about 24 weeks, about 8 weeks to about 36 weeks, about 8 weeks to about 48 weeks, about 8 weeks to about 60 weeks, about 12 weeks to about 20 weeks, about 12 weeks to about 28 weeks, about 12 weeks to about 40 weeks, about 12 weeks to about 52 weeks, about 12 weeks to about 72 weeks, about 16 weeks to about 24 weeks, about 16 weeks to about 36 weeks, about 16 weeks to about 48 weeks, about 16 weeks to about 60 weeks, about 20 weeks to about 28 weeks, about 20 weeks to about 40 weeks, about 20 weeks to about 52 weeks, about 20 weeks to about 72 weeks, about 24 weeks to about 36 weeks, about 24 weeks to about 48 weeks, about 24 weeks to about 60 weeks, about 28 weeks to about 40 weeks, about 28 weeks to about 52 weeks, about 28 weeks to about 72 weeks, about 36 weeks to about 48 weeks, about 36 weeks to about 60 weeks, about 40 weeks to about 44 weeks, about 40 weeks to about 52 weeks, about 40 weeks to about 72 weeks, about 44 weeks to about 52 weeks, about 44 weeks to about 72 weeks, about 48 weeks to about 60 weeks, about 52 weeks to about 72 weeks, or about 60 weeks to about 72 weeks. For example, total bilirubin can be reduced by about 50% to about 100% (e.g., about 50% to about 60%; about 50% to about 70%; about 50% to about 80%; about 50% to about 90%; about 60% to about 70%; about 60% to about 80%; about 60% to about 90%; about 60% to about 100%; about 70% to about 80%; about 70% to about 90%; about 70% to about 100%; about 80% to about 90%; about 80% to about 100%; or about 90% to about 100%) following administration of the ASBT inhibitor, or a pharmaceutically acceptable salt thereof.

In some embodiments, serum alkaline phosphatase (ALP) levels are improved following administration of the ASBT inhibitor, or a pharmaceutically acceptable salt thereof. In some embodiments, ALP levels are decreased following administration of the ASBT inhibitor, or a pharmaceutically acceptable salt thereof. In some embodiments, ALP levels are decreased about 50 U/L to about 175 U/L, about 50 U/L to about 150 U/L, about 50 U/L to about 125 U/L, or about 100 U/L to about 150 U/L from baseline following administration of the ASBT inhibitor, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, ALP levels can be reduced approximately 50%, approximately 60% or approximately 70% following administration of the ASBT inhibitor, or a pharmaceutically acceptable salt thereof, for at least 24 weeks.

In some embodiments, ALP levels are decreased about 50 U/L to about 175 U/L, about 50 U/L to about 150 U/L, about 50 U/L to about 125 U/L, about 50 U/L to about 75 U/L, about 75 U/L to about 175 U/L, about 75 U/L to about 150 U/L, about 75 U/L to about 125 U/L, about 75 U/L to about 100 U/L, about 100 U/L to about 175 U/L, about 100 U/L to about 150 U/L, about 100 U/L to about 125 U/L, or about 150 U/L to about 175 U/L from baseline following administration of the ASBT inhibitor, or a pharmaceutically acceptable salt thereof, for about 1 week to about 72 weeks (e.g., about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 16 weeks, about 4 weeks to about 24 weeks, about 4 weeks to about 40 weeks, about 4 weeks to about 52 weeks, about 4 weeks to about 72 weeks, about 8 weeks to about 16 weeks, about 8 weeks to about 24 weeks, about 8 weeks to about 36 weeks, about 8 weeks to about 48 weeks, about 8 weeks to about 60 weeks, about 12 weeks to about 20 weeks, about 12 weeks to about 28 weeks, about 12 weeks to about 40 weeks, about 12 weeks to about 52 weeks, about 12 weeks to about 72 weeks, about 16 weeks to about 24 weeks, about 16 weeks to about 36 weeks, about 16 weeks to about 48 weeks, about 16 weeks to about 60 weeks, about 20 weeks to about 28 weeks, about 20 weeks to about 40 weeks, about 20 weeks to about 52 weeks, about 20 weeks to about 72 weeks, about 24 weeks to about 36 weeks, about 24 weeks to about 48 weeks, about 24 weeks to about 60 weeks, about 28 weeks to about 40 weeks, about 28 weeks to about 52 weeks, about 28 weeks to about 72 weeks, about 36 weeks to about 48 weeks, about 36 weeks to about 60 weeks, about 40 weeks to about 44 weeks, about 40 weeks to about 52 weeks, about 40 weeks to about 72 weeks, about 44 weeks to about 52 weeks, about 44 weeks to about 72 weeks, about 48 weeks to about 60 weeks, about 52 weeks to about 72 weeks, or about 60 weeks to about 72 weeks. For example, ALP levels can be reduced by about 50%, about 60% or about 70% following administration of the ASBT inhibitor, or a pharmaceutically acceptable salt thereof.

In some embodiments, serum alanine aminotransferase (ALT) levels are improved following administration of the ASBT inhibitor, or a pharmaceutically acceptable salt thereof. In some embodiments, ALT levels are decreased following administration of the ASBT inhibitor, or a pharmaceutically acceptable salt thereof. In some embodiments, ALT levels are decreased about 50 U/L to about 175 U/L, about 50 U/L to about 150 U/L, about 50 U/L to about 125 U/L, or about 100 U/L to about 150 U/L from baseline following administration of the ASBT inhibitor, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, ALT levels can be reduced approximately 50%, approximately 60% or approximately 70% following administration of the ASBT inhibitor, or a pharmaceutically acceptable salt thereof, for at least 24 weeks.

In some embodiments, serum aspartate aminotransferase (AST) levels are improved following administration of the ASBT inhibitor, or a pharmaceutically acceptable salt thereof.

In some embodiments, ALT levels are decreased about 50 U/L to about 175 U/L, about 50 U/L to about 150 U/L, about 50 U/L to about 125 U/L, about 50 U/L to about 75 U/L, about 75 U/L to about 175 U/L, about 75 U/L to about 150 U/L, about 75 U/L to about 125 U/L, about 75 U/L to about 100 U/L, about 100 U/L to about 175 U/L, about 100 U/L to about 150 U/L, about 100 U/L to about 125 U/L, or about 150 U/L to about 175 U/L from baseline following administration of the ASBT inhibitor, or a pharmaceutically acceptable salt thereof, for about 1 week to about 72 weeks (e.g., about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 16 weeks, about 4 weeks to about 24 weeks, about 4 weeks to about 40 weeks, about 4 weeks to about 52 weeks, about 4 weeks to about 72 weeks, about 8 weeks to about 16 weeks, about 8 weeks to about 24 weeks, about 8 weeks to about 36 weeks, about 8 weeks to about 48 weeks, about 8 weeks to about 60 weeks, about 12 weeks to about 20 weeks, about 12 weeks to about 28 weeks, about 12 weeks to about 40 weeks, about 12 weeks to about 52 weeks, about 12 weeks to about 72 weeks, about 16 weeks to about 24 weeks, about 16 weeks to about 36 weeks, about 16 weeks to about 48 weeks, about 16 weeks to about 60 weeks, about 20 weeks to about 28 weeks, about 20 weeks to about 40 weeks, about 20 weeks to about 52 weeks, about 20 weeks to about 72 weeks, about 24 weeks to about 36 weeks, about 24 weeks to about 48 weeks, about 24 weeks to about 60 weeks, about 28 weeks to about 40 weeks, about 28 weeks to about 52 weeks, about 28 weeks to about 72 weeks, about 36 weeks to about 48 weeks, about 36 weeks to about 60 weeks, about 40 weeks to about 44 weeks, about 40 weeks to about 52 weeks, about 40 weeks to about 72 weeks, about 44 weeks to about 52 weeks, about 44 weeks to about 72 weeks, about 48 weeks to about 60 weeks, about 52 weeks to about 72 weeks, or about 60 weeks to about 72 weeks. For example, ALT levels can be reduced about 50%, about 60% or about 70% following administration of the ASBT inhibitor, or a pharmaceutically acceptable salt thereof.

In some embodiments, following administration of the ASBT inhibitor, or a pharmaceutically acceptable salt thereof, the subject exhibits a reduction of serum blood urea nitrogen (BUN). In some embodiments, BUN levels are decreased about 0.5 mg/dl to about 5.0 mg/dl, about 1 mg/dl to about 5.0 mg/dl, about 1.5 mg/dl to about 5.0 mg/dl, or about 2.0 mg/dl to about 5.0 mg/dl from baseline following administration of the ASBT inhibitor, or a pharmaceutically acceptable salt thereof, for at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, etc. For example, BUN levels can be reduced approximately 50%, approximately 60% or approximately 70% following administration of the ASBT inhibitor, or a pharmaceutically acceptable salt thereof, for at least 24 weeks.

In some embodiments, BUN levels are decreased about 0.5 mg/dl to about 5.0 mg/dl, about 0.5 mg/dl to about 4.0 mg/dL, about 0.5 mg/dl to about 3.0 mg/dl, about 0.5 mg/dl to about 2.0 mg/dl, about 0.5 mg/dL to about 1.5 mg/dl, about 1.0 mg/dL to about 5.0 mg/dl, about 1.0 mg/dL to about 4.0 mg/dl, about 1.0 mg/dl to about 3.0 mg/dl, about 1.0 mg/dl to about 2.0 mg/dl, about 1.0 mg/dl to about 1.5 mg/dl, about 1.5 mg/dl to about 5.0 mg/dl, about 1.5 mg/dl to about 4.0 mg/dl, about 1.5 mg/dl to about 3.0 mg/dl, about 1.5 mg/dL to about 2.0 mg/dl, about 2.0 mg/dl to about 5.0 mg/dL, about 2.0 mg/dL to about 4.0 mg/dl, about 2.0 mg/dl to about 3.0 mg/dl, about 3.0 mg/dl to about 5.0 mg/dL, about 3.0 mg/dl to about 4.0 mg/dl, or 4.0 mg/dl to about 5.0 mg/dl from baseline following administration of the ASBT inhibitor, or a pharmaceutically acceptable salt thereof, for about 1 week to about 72 weeks (e.g., about 1 week to about 4 weeks, about 1 week to about 8 weeks, about 1 week to about 12 weeks, about 1 week to about 16 weeks, about 1 week to about 20 weeks, about 1 week to about 24 weeks, about 1 week to about 36 weeks, about 1 week to about 40 weeks, about 1 week to about 48 weeks, about 1 week to about 52 weeks, about 1 week to about 60 weeks, about 4 weeks to about 8 weeks, about 4 weeks to about 16 weeks, about 4 weeks to about 24 weeks, about 4 weeks to about 40 weeks, about 4 weeks to about 52 weeks, about 4 weeks to about 72 weeks, about 8 weeks to about 16 weeks, about 8 weeks to about 24 weeks, about 8 weeks to about 36 weeks, about 8 weeks to about 48 weeks, about 8 weeks to about 60 weeks, about 12 weeks to about 20 weeks, about 12 weeks to about 28 weeks, about 12 weeks to about 40 weeks, about 12 weeks to about 52 weeks, about 12 weeks to about 72 weeks, about 16 weeks to about 24 weeks, about 16 weeks to about 36 weeks, about 16 weeks to about 48 weeks, about 16 weeks to about 60 weeks, about 20 weeks to about 28 weeks, about 20 weeks to about 40 weeks, about 20 weeks to about 52 weeks, about 20 weeks to about 72 weeks, about 24 weeks to about 36 weeks, about 24 weeks to about 48 weeks, about 24 weeks to about 60 weeks, about 28 weeks to about 40 weeks, about 28 weeks to about 52 weeks, about 28 weeks to about 72 weeks, about 36 weeks to about 48 weeks, about 36 weeks to about 60 weeks, about 40 weeks to about 44 weeks, about 40 weeks to about 52 weeks, about 40 weeks to about 72 weeks, about 44 weeks to about 52 weeks, about 44 weeks to about 72 weeks, about 48 weeks to about 60 weeks, about 52 weeks to about 72 weeks, or about 60 weeks to about 72 weeks. For example, BUN levels can be reduced approximately 50%, approximately 60% or approximately 70% following administration of the ASBT inhibitor, or a pharmaceutically acceptable salt thereof, for at least 24 weeks.

In some embodiments, following administration of the ASBT inhibitor, or a pharmaceutically acceptable salt thereof, the subject exhibits a reduction in urinary neutrophil gelatinase-associated lipocalin (NGAL). In some embodiments, the subject exhibits a reduction in urinary NGAL between about 5% and about 100%, such as between about 10% and about 100%, between about 15% and about 100%, between about 25% and about 100%, between about 50% and about 100%, between about 75% and about 100%, between about 10% and about 75%, between about 25% and about 75% or between about 50% and about 75%. In some embodiments, the subject exhibits a reduction in urinary NGAL of at least about 50% (e.g., at least about 55%; at least about 60; at least about 65%; at least about 70%; at least about 75%; at least about 80%; at least about 85%; at least about 90%; or at least about 95%). In some embodiments, the subject exhibits a reduction in urinary NGAL of at least about 60%, at least about 70%, at least about 80%, or at least about 90%. In some embodiments, the subject exhibits a reduction in urinary NGAL of about 60%, about 70%, about 80%, or about 90%.

In some embodiments, following administration of the ASBT inhibitor, or a pharmaceutically acceptable salt thereof, the subject exhibits a reduction in urinary kidney injury molecule-1 (KIM-1). In some embodiments, the subject exhibits a reduction in urinary KIM-1 of between about 5% and about 100%, such as between about 10% and about 100%, between about 15% and about 100%, between about 25% and about 100%, between about 50% and about 100%, between about 75% and about 100%, between about 10% and about 75%, between about 25% and about 75% or between about 50% and about 75%. In some embodiments, the subject exhibits a reduction in urinary KIM-1 of at least 50% (e.g., at least 55%; at least 60; at least 65%; at least 70%; at least 75%; at least 80%; at least 85%; at least 90%; or at least 95%). In some embodiments, the subject exhibits a reduction in urinary KIM-1 of at least 60%, at least 70%, at least 80%, or at least 90%. In some embodiments, the subject exhibits a reduction in urinary KIM-1 of about 60%, about 70%, about 80%, or about 90%.

In some embodiments, the ASBT inhibitor is administered orally. Because ASBT is predominantly expressed in the ileum (where it is often referred to as IBAT), ASBT inhibitors need not be systemically available. Indeed, the systemic absorption of the vast majority of known ASBT inhibitors is low, such as less than 10%. However, since ASBT is also expressed in the proximal tubule cells of the kidneys, systemically available ASBT inhibitors may also inhibit the reuptake of bile acids in the kidneys. It is believed that this may lead to increased levels of bile acids in urine, and to an increased removal of bile acids from the body via the urine. Consequently, systemically available ASBT inhibitors that exert their effect not only in the ileum but also in the kidneys are expected to lead to a greater reduction of bile acid levels than non-systemically available ASBT inhibitors that only exert their effect in the ileum. Targeting renal ASBT may thus be an alternative or additional means of increasing bile acid excretion and reducing bile acid load in serum, the liver, and the kidneys. In some embodiments, therefore, the ASBT inhibitor Is systemically available. In some embodiments, the systemic absorption of the ASBT inhibitor is between about 10% and about 100%, such as between about 10% and about 75%, between about 10% and 50%, between about 10% and about 25%, between about 25% and about 100%, between about 25% and about 75%, between about 25% and about 50%, between about 50% and about 100%, between about 50% and about 75%, or between about 75% and about 100%. In some embodiments, the systemic absorption of the ASBT inhibitor is greater than about 10%, such as greater than about 15%, such as greater than about 20%, such as greater than about 25% or such as greater than about 30%. In some embodiments, the systemic absorption of the ASBT inhibitor is about 10%, about 15%, about 20%, about 25%, about 30%, about 40%, or about 50% or greater.

In some embodiments, the ASBT inhibitor is administered subcutaneously. It has been found that subcutaneous administration of an ASBT inhibitor may result in a high bioavailability, with a constant exposure lasting for more than 24 hours. Subcutaneous administration of an ASBT inhibitor may therefore provide a different and possibly longer lasting bile acid modulating effect than oral administration of the ASBT inhibitor. Such an effect may be useful in the treatment of diseases wherein a stronger inhibition of the bile acid circulation is required or when oral administration is not likely to provide benefit (i.e., when bile flow is blocked).

In some embodiments, the oral administration of an ASBT inhibitor is combined with the subcutaneous administration of an ASBT inhibitor. Such combined treatment may have an additive or synergistic effect, and may result in the excretion of even larger amounts of bile acids. Examples of non-systemically available ASBT inhibitors include, but are not limited to, elobixibat, odevixibat, maralixibat, volixibat and linerixibat. The systemic absorption following oral administration of these ASBT inhibitors is less than 10%. Further examples of suitable ASBT inhibitors are disclosed in e.g., WO 2019/234077, WO 2020/161216, WO 2020/161217, WO 2021/110884, WO 2021/110885, WO 2021/110886, WO 2021/110887 and WO 2022/029101.

In some embodiments, the patient does not respond to treatment with an orally administered, non-systemically available ASBT inhibitor. As the subcutaneous administration of an ASBT inhibitor leads to modulation of the renal ASBT, it is believed that subcutaneous administration of an ASBT inhibitor may result in a stronger ASBT modulating effect than oral administration of said compound.

In some embodiments, the patient does not tolerate treatment with an orally administered, non-systemically available ASBT inhibitor, for instance when the patient experiences severe side effects such as severe diarrhoea. Because subcutaneous administration of an ASBT inhibitor also results in modulation of the renal ASBT, bile acids are excreted not only in stools but also in urine. This is expected to lead to a reduction in the incidence of diarrhoea.

### Combination Therapy

In one aspect of the invention, the ASBT inhibitor, or pharmaceutically acceptable salts thereof, is administered in combination with at least one other therapeutically active agent, such as with one, two, three or more other therapeutically active agents. The ASBT inhibitor, or a pharmaceutically acceptable salt thereof, and the at least one other therapeutically active agent may be administered simultaneously, sequentially or separately. Therapeutically active agents that are suitable for combination with an ASBT inhibitor include, but are not limited to, known active agents that are useful in the treatment of any of the diseases and disorders discussed herein.

In one embodiment, the ASBT inhibitor, or pharmaceutically acceptable salts thereof, is administered in combination with another ASBT inhibitor. Suitable ASBT inhibitors are disclosed in WO 93/16055, WO 94/18183, WO 94/18184, WO 96/05188, WO 96/08484, WO 96/16051, WO 97/33882, WO 98/03818, WO 98/07449, WO 98/40375, WO 99/35135, WO 99/64409, WO 99/64410, WO 00/01687, WO 00/47568, WO 00/61568, WO 00/38725, WO 00/38726, WO 00/38727, WO 00/38728, WO 00/38729, WO 01/66533, WO 01/68096, WO 02/32428, WO 02/50051, WO 03/020710, WO 03/022286, WO 03/022825, WO 03/022830, WO 03/061663, WO 03/091232, WO 03/106482, WO 2004/006899, WO 2004/076430, WO 2007/009655, WO 2007/009656, WO 2011/137135, WO 2019/234077, WO 2020/161216, WO 2020/161217, WO 2021/110884, WO 2021/110885, WO 2021/110886, WO 2021/110887, WO 2022/029101, DE 19825804, EP 864582, EP 489423, EP 549967, EP 573848, EP 624593, EP 624594, EP 624595, EP 624596, EP 0864582, EP 1173205, EP 1535913 and EP 3210977, all of which are incorporated herein by reference in their entireties. Particular examples of suitable ASBT inhibitors include 1,1-dioxo-3,3-dibutyl-5-phenyl-7-methylthio-8-(N-{(R)-1′-phenyl-1′-[N′-(carboxymethyl)carbamoyl]-methyl}carbamoylmethoxy)-2,3,4,5-tetrahydro-1,5-benzothiazepine (elobixibat) and 1,1-dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine (odevixibat), 1-{[4-({4-[(4R,5R)-3,3-dibutyl-7-(dimethylamino)-4-hydroxy-1, 1-dioxo-2,3,4,5-tetrahydro-1H-1λ⁶-benzothiepin-5-yl]phenoxy}methyl)phenyl]methyl}-1,4-diazabicyclo[2.2.2]octan-1-ium chloride (maralixibat), N-(3-O-benzyl-6-O-sulfo-β-D-glucopyranosyl)-N′-{3-[(3S,4R,5R)-3-butyl-7-(dimethylamino)-3-ethyl-4-hydroxy-1,1-dioxo-2,3,4,5-tetrahydro-1H-1λ⁶-benzothiepin-5-yl]phenyl}urea (volixibat) and 3-({[(3R,5R)-3-butyl-3-ethyl-7-methoxy-1,1-dioxo-5-phenyl-2,3,4,5-tetrahydro-1H-1λ⁶,4-benzothiazepin-8-yl]methyl}amino)pentanedioic acid (linerixibat).

Some ASBT inhibitors may show a higher free fraction in plasma. In some embodiments, the free fraction is from about 0% up to about 100%, such as from about 0% up to about 75%, about 0% up to about 50%, about 0% up to about 25%, about 0% up to about 10%, or about 0% up to about 5%. In some embodiments, the free fraction is from about 0.1% to about 100%, e.g., about 0.1% to about 75%, about 0.1% to about 50%, about 0.1% to about 25%, about 0.1% to about 15%, about 0.1% to about 10%, about 0.1% to about 5%, about 0.1% to about 1%, about 1% to about 100%, about 1% to about 75%, about 1% to about 50%, about 1% to about 25%, about 1% to about 15%, about 1% to about 10%, about 1% to about 5%, about 5% to about 100%, about 5% to about 75%, about 5% to about 50%, about 5% to about 25%, about 5% to about 15%, about 5% to about 10%, about 10% to about 100%, about 10% to about 75%, about 10% to about 50%, about 10% to about 25%, about 10% to about 15%, about 15% to about 100%, about 15% to about 75%, about 15% to about 50%, about 15% to about 25%, about 25% to about 100%, about 25% to about 75%, or about 25% to about 50%. In some embodiments, the free fraction is greater than about 0.2%, such as greater than about 0.4%, such as greater than about 0.6%, such as greater than about 0.8%, such as greater than about 1.0%, such as greater than about 1.25%, such as greater than about 1.5%, such as greater than about 1.75%, such as greater than about 2.0%, such as greater than about 2.5%, such as greater than about 3%, such as greater than about 4%, such as greater than about 5%, such as greater than about 7.5%, such as greater than about 10%, or such as greater than about 20%.

Some ASBT inhibitors may be excreted in urine. In some embodiments, the fraction of the compound that is excreted in urine is from about 0% to about 100%, or such as from about 0% to about 75%, about 0% to about 50%, about 0% up to about 25%, about 0% up to about 10%, or about 0% up to about 5%. In some embodiments, the fraction of the compound that is excreted in urine is from about 0.1% to about 100%, e.g., about 0.1% to about 75%, about 0.1% to about 50%, about 0.1% to about 25%, about 0.1% to about 15%, about 0.1% to about 10%, about 0.1% to about 5%, about 0.1% to about 1%, about 1% to about 100%, about 1% to about 75%, about 1% to about 50%, about 1% to about 25%, about 1% to about 15%, about 1% to about 10%, about 1% to about 5%, about 5% to about 100%, about 5% to about 75%, about 5% to about 50%, about 5% to about 25%, about 5% to about 15%, about 5% to about 10%, about 10% to about 100%, about 10% to about 75%, about 10% to about 50%, about 10% to about 25%, about 10% to about 15%, about 15% to about 100%, about 15% to about 75%, about 15% to about 50%, about 15% to about 25%, about 25% to about 100%, about 25% to about 75%, or about 25% to about 50%. In some embodiments, the fraction of the compound that is excreted in urine is greater than about 0.2%, such as greater than about 0.4%, such as greater than about 0.6%, such as greater than about 0.8%, such as greater than about 1.0%, such as greater than about 2%, such as greater than about 3%, such as greater than about 5%, such as greater than about 7.5%, such as greater than about 10%, such as greater than about 15%, such as greater than about 20%, such as greater than about 30%, or such as greater than about 50%.

Following absorption from the intestine, some ASBT inhibitors may be circulated via the enterohepatic circulation. In some embodiments, the fraction of the compound that is circulated via the enterohepatic circulation is from about 0% to about 100%, such as from about 0% to about 75%, about 0% to about 50%, about 0% up to about 25%, about 0% up to about 10%, or about 0% up to about 5%. In some embodiments, the fraction of the compound that is circulated via the enterohepatic circulation is from about 0.1% to about 100%, e.g., about 0.1% to about 75%, about 0.1% to about 50%, about 0.1% to about 25%, about 0.1% to about 15%, about 0.1% to about 10%, about 0.1% to about 5%, about 0.1% to about 1%, about 1% to about 100%, about 1% to about 75%, about 1% to about 50%, about 1% to about 25%, about 1% to about 15%, about 1% to about 10%, about 1% to about 5%, about 5% to about 100%, about 5% to about 75%, about 5% to about 50%, about 5% to about 25%, about 5% to about 15%, about 5% to about 10%, about 10% to about 100%, about 10% to about 75%, about 10% to about 50%, about 10% to about 25%, about 10% to about 15%, about 15% to about 100%, about 15% to about 75%, about 15% to about 50%, about 15% to about 25%, about 25% to about 100%, about 25% to about 75%, or about 25% to about 50%. In some embodiments, the fraction of the compound that is circulated via the enterohepatic circulation is greater than about 0.1%, such as greater than about 0.2%, such as greater than about 0.3%, such as greater than about 0.5%, such as greater than about 1.0%, such as greater than about 1.5%, such as greater than about 2%, such as greater than about 3%, such as greater than about 5%, such as greater than about 7%, such as greater than about 10%, such as greater than about 15%, such as greater than about 20%, such as greater than about 30% or such as greater than about 50%.

Some ASBT inhibitors may cause renal excretion of bile salts. In some embodiments, the fraction of circulating bile acids that is excreted by the renal route is from about 0% to about 100%, such as from about 0% to about 75%, about 0% to about 50%, about 0% up to about 25%, about 0% up to about 10%, or about 0% up to about 5%. In some embodiments, the fraction of the circulating bile acids that is excreted by the renal route is from about 0.1% to about 100%, e.g., about 0.1% to about 75%, about 0.1% to about 50%, about 0.1% to about 25%, about 0.1% to about 15%, about 0.1% to about 10%, about 0.1% to about 5%, about 0.1% to about 1%, about 1% to about 100%, about 1% to about 75%, about 1% to about 50%, about 1% to about 25%, about 1% to about 15%, about 1% to about 10%, about 1% to about 5%, about 5% to about 100%, about 5% to about 75%, about 5% to about 50%, about 5% to about 25%, about 5% to about 15%, about 5% to about 10%, about 10% to about 100%, about 10% to about 75%, about 10% to about 50%, about 10% to about 25%, about 10% to about 15%, about 15% to about 100%, about 15% to about 75%, about 15% to about 50%, about 15% to about 25%, about 25% to about 100%, about 25% to about 75%, or about 25% to about 50%. In some embodiments, the fraction of circulating bile acids that is excreted by the renal route is greater than about 1%, such as greater than about 2%, such as greater than about 5%, such as greater than about 7%, such as greater than about 10%, such as greater than about 15%, such as greater than about 20%, or such as greater than about 25%.

Some ASBT inhibitors may show improved or optimal permeability. The permeability may be measured in Caco2 cells, and values are given as Papp (apparent permeability) values in cm/s. In some embodiments, the permeability is between about 0.01×10⁻⁶ cm/s and about 50×10⁻⁶ cm/s, such as between about 0.05×10⁻⁶ cm/s and about 40×10⁻⁶ cm/s, or such as between about 0.1×10⁻⁶ cm/s and about 30×10⁻⁶ cm/s. In some embodiments, the permeability is greater than at least about 0.1×10⁻⁶ cm/s, such as greater than about 0.2×10⁻⁶ cm/s, such as greater than about 0.4×10⁻⁶ cm/s, such as greater than about 0.7×10⁻⁶ cm/s, such as greater than about 1.0×10⁻⁶ cm/s, such as greater than about 2×10⁻⁶ cm/s, such as greater than about 3×10⁻⁶ cm/s, such as greater than about 5×10⁻⁶ cm/s, such as greater than about 7×10⁻⁶ cm/s, such as greater than about 10×10⁻⁶ cm/s, such as greater than about 15×10⁻⁶ cm/s.

Some ASBT inhibitors may show an improved or optimal bioavailability. The oral bioavailability may be between about 0% and about 100%, such as between about 1% and about 100%, about 1% and about 90%, about 1% and about 80%, about 1% and about 75%, about 1% and about 60%, about 1% and about 50%, about 1% and about 40%, about 1% and about 30%, about 1% and about 20%, about 1% and about 10%, about 2% and about 50%, about 10% and about 100%, about 10% and about 90%, about 10% and about 80%, about 10% and about 75%, about 10% and about 60%, about 10% and about 50%, about 10% and about 40%, about 10% and about 30%, about 10% and about 20%, about 20% and about 100%, about 20% and about 90%, about 20% and about 80%, about 20% and about 75%, about 20% and about 60%, about 20% and about 50%, about 20% and about 40%, about 20% and about 30%, about 30% and about 100%, about 30% and about 90%, about 30% and about 80%, about 30% and about 75%, about 30% and about 60%, about 30% and about 50%, about 30% and about 40%, about 40% and about 100%, about 40% and about 90%, about 40% and about 80%, about 40% and about 75%, about 40% and about 60%, about 40% and about 50%, about 50% and about 100%, about 50% and about 90%, about 50% and about 80%, about 50% and about 75%, about 50% and about 60%, about 60% and about 100%, about 60% and about 90%, about 60% and about 80%, about 60% and about 75%, about 75% and about 100%, about 75% and about 90%, about 75% and about 80%, about 80% and about 100%, about 80% and about 90%, or about 90% and about 100%. In some embodiments, the oral bioavailability is greater than about 5%, such as greater than about 7%, such as greater than about 10%, such as greater than about 15%, such as greater than about 20%, such as greater than about 30%, such as greater than about 40%, such as greater than about 50%, such as greater than about 60%, such as greater than about 70% or such as greater than about 80%. In other embodiments, the oral bioavailability is between about 10 and about 90%, such as between about 20 and about 80%, such as between about 30 and about 70% or such as between about 40 and about 60%.

### Formulations

The ASBT inhibitor may be administered as a pharmaceutical composition comprising a therapeutically effective amount of the ASBT inhibitor or a pharmaceutically acceptable salt thereof, and one or more pharmaceutically acceptable excipients. In general, pharmaceutical compositions may be prepared in a conventional manner using conventional excipients. The pharmaceutical composition may be in a form that is suitable for oral administration, for parenteral injection (including intravenous, subcutaneous, intramuscular and intravascular injection), for topical administration of for rectal administration.

In some embodiments, the pharmaceutical composition is in a form that is suitable for oral administration, such as a tablet or a capsule. Such formulations may comprise, in addition to the ASBT inhibitor, excipients such as fillers, binders, disintegrants, glidants and lubricants.

Examples of suitable fillers include, but are not limited to, dicalcium phosphate dihydrate, calcium sulfate, lactose (such as lactose monohydrate), sucrose, mannitol, sorbitol, cellulose, microcrystalline cellulose, dry starch, hydrolyzed starches and pregelatinized starch.

Examples of suitable binders include, but are not limited to, starch, pregelatinized starch, gelatin, sugars (such as sucrose, glucose, dextrose, lactose and sorbitol), polyethylene glycol, waxes, natural and synthetic gums (such as acacia gum and tragacanth gum), sodium alginate, cellulose derivatives (such as hydroxypropylmethylcellulose (or hypromellose), hydroxypropylcellulose and ethylcellulose) and synthetic polymers (such as acrylic acid and methacrylic acid copolymers, methacrylic acid copolymers, methyl methacrylate copolymers, aminoalkyl methacrylate copolymers, polyacrylic acid/polymethacrylic acid copolymers and polyvinylpyrrolidone (povidone)).

Examples of suitable disintegrants include, but are not limited to, dry starch, modified starch (such as (partially) pregelatinized starch, sodium starch glycolate and sodium carboxymethyl starch), alginic acid, cellulose derivatives (such as sodium carboxymethylcellulose, hydroxypropyl cellulose, and low substituted hydroxypropyl cellulose (L-HPC)) and cross-linked polymers (such as carmellose, croscarmellose sodium, carmellose calcium and cross-linked PVP (crospovidone)).

Examples of suitable glidants and lubricants include, but are not limited to, talc, magnesium stearate, calcium stearate, stearic acid, glyceryl behenate, colloidal silica, aqueous silicon dioxide, synthetic magnesium silicate, fine granulated silicon oxide, starch, sodium lauryl sulfate, boric acid, magnesium oxide, waxes (such as carnauba wax), hydrogenated oil, polyethylene glycol, sodium benzoate, polyethylene glycol, and mineral oil.

The pharmaceutical composition may be conventionally coated with one or more coating layers. Enteric coating layers or coating layers for delayed or targeted release of the compound of formula (I), or pharmaceutically acceptable salts thereof, are also contemplated. The coating layers may comprise one or more coating agents, and may optionally comprise plasticizers and/or pigments (or colorants).

Example of suitable coating agents include, but are not limited to, cellulose-based polymers (such as ethylcellulose, hydroxypropylmethylcellulose (or hypromellose), hydroxypropylcellulose, cellulose acetate phthalate, cellulose acetate succinate, hydroxypropyl methylcellulose acetate succinate and hydroxypropyl methylcellulose phthalate), vinyl-based polymers (such as polyvinyl alcohol) and polymers based on acrylic acid and derivatives thereof (such as acrylic acid and methacrylic acid copolymers, methacrylic acid copolymers, methyl methacrylate copolymers, aminoalkyl methacrylate copolymers, polyacrylic acid/polymethacrylic acid copolymers).

Examples of suitable plasticizers include, but are not limited to, triethyl citrate, glyceryl triacetate, tributyl citrate, diethyl phthalate, acetyl tributyl citrate, dibutyl phthalate, dibutyl sebacate and polyethylene glycol.

Examples of suitable pigments include, but are not limited to, titanium dioxide, iron oxides (such as yellow, brown, red or black iron oxides) and barium sulfate.

In some embodiments, the pharmaceutical composition is in a form that is suitable for parenteral administration (e.g., subcutaneous administration), such as a liquid (aqueous) formulation. Such formulations may comprise, in addition to the ASBT inhibitor, solubilizing and stabilizing excipients such as salts (e.g., saline), buffers, surfactants, cosolvents, antioxidants and preservatives.

Buffers may include salts such as phosphate, citrate, acetate, gluconate, lactate, tartrate, aspartate, glutamate and phthalate, or the corresponding acid forms thereof, as well as histidine or Tris (tris(hydroxymethyl)aminomethane). The pH of the liquid formulation is within the range of about 4 to about 9, more preferably within the range of about 5 to about 8, and even more preferably within the range of about 6 to 7.

The surfactant may be a cationic surfactant, an anionic surfactant or a nonionic surfactant. Examples of cationic surfactants include, but are not limited to, cetyltrimethylammonium bromide (cetrimonium bromide) and cetylpyridinium chloride. Examples of anionic surfactants include, but are not limited to, sodium dodecyl sulfate (sodium lauryl sulfate) and ammonium dodecyl sulfate (ammonium lauryl sulfate). Examples of nonionic surfactants include, but are not limited to, glycerol monooleate, glycerol monostearate, polyoxyl castor oil (Cremophor EL), poloxamers (e.g., poloxamer 407 or 188), polysorbate 80 and sorbitan esters (Tween). In a preferred embodiment, the surfactant is a cationic surfactant.

Examples of suitable cosolvents include, but are not limited to, ethanol, propylene glycol, polyethylene glycol 400 (PEG 400), N-methylpyrrolidone (NMP), dimethyl sulfoxide (DMSO), and N,N-dimethylacetamide (DMA).

Examples of suitable antioxidants include, but are not limited to, butylhydroxytoluene (BHT), ascorbyl palmitate, propyl gallate and ascorbic acid, and combinations thereof.

Examples of suitable preservatives include, but are not limited to, phenol, benzyl alcohol, methyl paraben, ethyl paraben, propyl paraben, ethylenediaminetetraacetic acid (EDTA), potassium sorbate and sodium benzoate, and combinations thereof.

In some embodiments, the concentration of the ASBT inhibitor in a liquid formulation is from about 0.001 to about 30 mg/mL. In some embodiments, the concentration of the ASBT inhibitor in a liquid formulation is from about 0.01 to about 10 mg/mL, such as from about 0.01 to about 5 mg/ml, about 0.01 to about 2 mg/ml, about 0.01 to about 1.5 mg/mL, about 0.01 to about 1.0 mg/ml, about 1 to about 10 mg/ml, about 1 to about 5 mg/mL, about 1 to about 2 mg/ml, about 1 to about 1.5 mg/ml, about 2 to about 10 mg/ml, about 2 to about 5 mg/ml, about 5 to about 10 mg/ml; or such as from about 10 to about 30 mg/mL, such as from about 10 to about 20 mg/ml, or such as from about 20 to about 30 mg/mL. In some embodiments, the concentration of the ASBT inhibitor in a liquid formulation is about 0.2 mg/ml, about 0.3 mg/ml, about 0.4 mg/ml, about 0.5 mg/ml, about 0.6 mg/ml, about 0.7 mg/mL, about 0.8 mg/ml, about 0.9 mg/ml, about 1.0 mg/mL, about 1.2 mg/mL, about 1.4 mg/mL, about 1.6 mg/mL, about 1.8 mg/ml or about 2.0 mg/mL.

In some embodiments, the pharmaceutical composition optionally comprises one or more additional therapeutic agents as described herein.

### Dose and Frequency of Administration

The dosage required for the therapeutic or prophylactic treatment will depend on the route of administration, the severity of the disease, the age and weight of the patient and other factors normally considered by the attending physician, when determining the appropriate regimen and dosage level for a particular patient.

The amount of the ASBT inhibitor to be administered will vary for the patient being treated, and may vary from about 1 μg/kg of body weight to about 50 mg/kg of body weight per day. In some embodiments, a patient is administered about 1 μg/kg/day to about 50 mg/kg/day of the ASBT inhibitor, or a pharmaceutically acceptable salt thereof, e.g., about 1 μg/kg/day to about 25 mg/kg/day, about 1 μg/kg/day to about 10 mg/kg/day, about 1 μg/kg/day to about 5 mg/kg/day, about 1 μg/kg/day to about 1 mg/kg/day, about 1 μg/kg/day to about 800 μg/kg/day, about 1 μg/kg/day to about 400 μg/kg/day, about 1 μg/kg/day to about 200 μg/kg/day, about 1 μg/kg/day to about 160 μg/kg/day, about 1 μg/kg/day to about 140 μg/kg/day, about 1 μg/kg/day to about 120 μg/kg/day, about 1 μg/kg/day to about 100 μg/kg/day, about 1 μg/kg/day to about 75 μg/kg/day, about 1 μg/kg/day to about 50 μg/kg/day, about 1 μg/kg/day to about 25 μg/kg/day, about 1 μg/kg/day to about 10 μg/kg/day, about 50 μg/kg/day to about 50 mg/kg/day, about 50 μg/kg/day to about 25 mg/kg/day, about 50 μg/kg/day to about 10 mg/kg/day, about 50 μg/kg/day to about 5 mg/kg/day, about 50 μg/kg/day to about 1 mg/kg/day, about 50 μg/kg/day to about 800 μg/kg/day, about 50 μg/kg/day to about 400 μg/kg/day, about 50 μg/kg/day to about 200 μg/kg/day, about 50 μg/kg/day to about 160 μg/kg/day, about 50 μg/kg/day to about 140 μg/kg/day, about 50 μg/kg/day to about 120 μg/kg/day, about 50 μg/kg/day to about 100 μg/kg/day, about 50 μg/kg/day to about 75 μg/kg/day, about 200 μg/kg/day to about 50 mg/kg/day, about 200 μg/kg/day to about 25 mg/kg/day, about 200 μg/kg/day to about 10 mg/kg/day, about 200 μg/kg/day to about 5 mg/kg/day, about 200 μg/kg/day to about 1 mg/kg/day, about 200 μg/kg/day to about 800 μg/kg/day, about 200 μg/kg/day to about 400 μg/kg/day, about 400 μg/kg/day to about 50 mg/kg/day, about 400 μg/kg/day to about 25 mg/kg/day, about 400 μg/kg/day to about 10 mg/kg/day, about 400 μg/kg/day to about 5 mg/kg/day, about 400 μg/kg/day to about 1 mg/kg/day, about 400 μg/kg/day to about 800 μg/kg/day, about 800 μg/kg/day to about 50 mg/kg/day, about 800 μg/kg/day to about 25 mg/kg/day, about 800 μg/kg/day to about 10 mg/kg/day, about 800 μg/kg/day to about 5 mg/kg/day, about 800 μg/kg/day to about 1 mg/kg/day, about 5 mg/kg/day to about 50 mg/kg/day, about 5 mg/kg/day to about 25 mg/kg/day, or about 5 mg/kg/day to about 10 mg/kg/day of the ASBT inhibitor, or a pharmaceutically acceptable salt thereof.

A unit dose form, such as a tablet or capsule, will usually contain about 0.1 to about 250 mg of active ingredient, such as about 0.1 to about 150 mg, about 0.1 to about 100 mg, about 0.1 to about 75 mg, about 0.1 to about 50 mg, about 0.1 to about 20 mg, e.g. about 0.2 mg, about 0.4 mg, about 0.6 mg, about 1.2 mg, about 2.5 mg, about 5 mg, about 10 mg, or about 15 mg. In some embodiments, a unit dose form, such as a tablet or capsule, contains about 10 mg to about 250 mg, 10 mg to about 200 mg, 10 mg to about 150 mg, 10 mg to about 100 mg, 10 mg to about 100 mg, 10 mg to about 75 mg, 10 mg to about 50 mg, about 50 mg to about 250 mg, 50 mg to about 200 mg, 50 mg to about 150 mg, 50 mg to about 100 mg, 50 mg to about 100 mg, 50 mg to about 75 mg, about 75 mg to about 250 mg, 75 mg to about 200 mg, 75 mg to about 150 mg, 75 mg to about 100 mg, 75 mg to about 100 mg, about 100 mg to about 250 mg, 100 mg to about 200 mg, 100 mg to about 150 mg, about 150 mg to about 250 mg, 150 mg to about 200 mg, or about 200 mg to about 250 mg. The daily dose can be administered as a single dose or divided into one, two, three or more unit doses. An orally administered daily dose of a bile acid modulator is preferably within about 0.1 to about 250 mg, more preferably within about 0.1 to about 100 mg, such as within about 0.1 to about 20 mg, about 0.1 to about 15 mg, about 0.1 to about 10 mg, about 0.1 to about 9 mg, about 0.1 to about 8 mg, about 1 to about 7 mg, about 1 to about 6 mg, about 0.1 to about 5 mg, about 0.1 to about 4 mg, about 0.1 to about 3 mg, about 0.1 to about 2 mg, about 0.1 to about 1 mg, about 1 to about 10 mg, about 1 to about 9 mg, about 1 to about 8 mg, about 1 to about 7 mg, about 1 to about 6 mg, about 1 to about 5 mg, about 1 to about 4 mg, about 1 to about 3 mg, about 1 to about 2 mg, about 2 to about 5 mg, about 2 to about 8 mg, about 2 to about 10 mg, about 5 to about 10 mg, about 5 to about 8 mg, or about 8 to about 10 mg.

As used herein, the terms “treatment”, “treat” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “subject,” “individual,” or “patient,” used interchangeably, refer to any animal, including mammals such as mice, rats, other rodents, rabbits, dogs, cats, swine, cattle, sheep, horses, primates, and humans. In some embodiments, the subject is a human.

As used herein, the term “baseline” refers to information obtained prior to the first administration of the drug or intervention of interest (e.g., at the beginning of a study) or an initial known value that is used for comparison with later data. Baseline values are taken at time “zero” (i.e., before subjects in a study receive the drug or intervention of interest or placebo).

As used herein, the term “normalized” refers to age-specific values that are within a range corresponding to a healthy individual (i.e., normal or normalized values).

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally, the term “about” refers to the indicated value of the variable and to all values of the variable that are within the experimental error of the indicated value (e.g., within the 95% confidence interval for the mean) or within 10 percent of the indicated value, whichever is greater.

### EXAMPLES

ASBT inhibitor compounds 1 to 5 were used in the experiments described herein. The potency (hIBAT IC₅₀), permeability and bioavailability of these compounds is shown in table 1 below. Values were determined using the assays described in e.g. WO 2020/161217.

TABLE 1

| | | Permeability | | |
|---|---|---|---|---|
| | | (Caco-2) | | |
| | hIBAT | Pₐₚₚ A2B | Pₐₚₚ B2A | |
| | IC₅₀ | (×10₋₆ | (×10₋₆ | Bioavailability |
| Compound | (nM) | cm/sec) | cm/sec) | (%) |
| Compound 1 | 21 | | | 44 (C57BL/6) |
| Compound 2 | 7 | 9.2 | 0.9 | 34 (C57 BL/6); 59 (CD1) |
| | | | | 98 (Na₊ salt; rat) |
| Compound 3 | 0.7 | 29.3 | 25.3 | |
| Compound 4 | 7.7 | | | |
| Compound 5 | 2.4 | 8.7 | 12.9 | |

### Example 1

### Mouse Model of Cholemic Nephropathy—Bile Duct Litigation (BDL) in Mice

The time-resolved events in the liver and the kidney after ligation of the extrahepatic common bile duct were evaluated.

Male mice (C57BL/6n) were used. After completion of quarantine and acclimatization period, animals were randomized based on body weight into experimental groups (n=5 mice per group and time point). On day 0, animals underwent bile duct ligation or sham surgery. Animals were euthanized after 1 or 3 days or after 1, 3, 6, 9 or 12 weeks, and samples (bile, blood, urine, liver and kidney tissues) were collected and analysed.

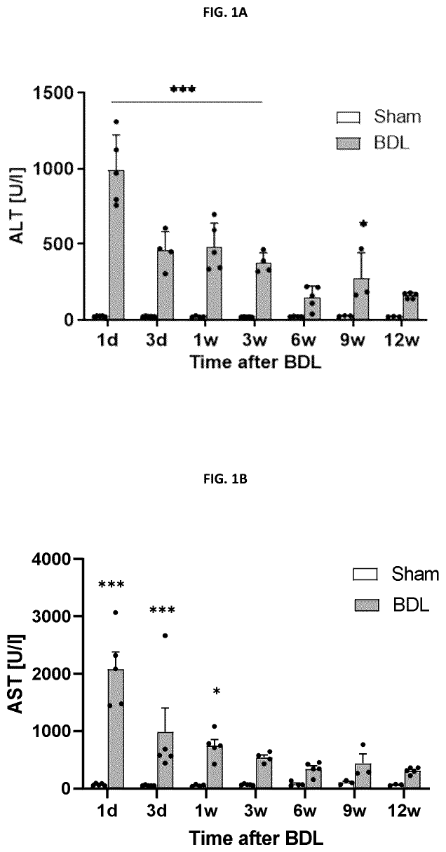

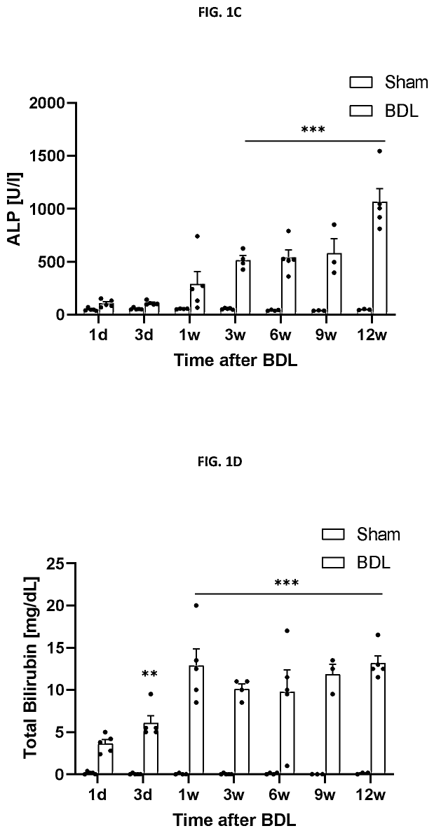

BDL led to excessive accumulation of bile in the gallbladder, and to increased serum ALT and AST which peaked on day 1 and decreased later when the liver adapted to cholestasis, but remained above control values (FIGS. 1A and 1B). Alkaline phosphatase (ALP) enzyme activity showed time-dependent elevation after BDL (FIG. 1C).

Analysis of the expression of bile acid transporters showed that ASBT was strongly downregulated after BDL. FIGS. 2A-2D show the compensatory alterations of bile acid transporter expression during 12 weeks following BDL or sham surgery. FIG. 2A: sinusoidal uptake transporter NTCP; FIG. 2B: sinusoidal uptake transporter Cyp7a1; FIG. 2C: apical transporter Bsep; FIG. 2D: sinusoidal export transporter MRP4. FIGS. 3A-3E show the expression of the apical uptake transporters ASBT (FIG. 3A) and OATP1a1 (FIG. 3B), the apical export transporter MRP4 (FIG. 3C) and the basolateral export transporters MRP3 (FIG. 3D) and OSTα (FIG. 3E) during 12 weeks following BDL or sham surgery.

A similar study was performed using female mice.

### Example 2

### Effect of ASBT Inhibition in BDL Mice

The short-term effect of oral administration of Compound 1 to BDL mice on the urinary excretion of bile acids was evaluated.

Female mice (C57BL/6n) were used. After completion of quarantine and acclimatization period, animals were randomized based on body weight into experimental groups (n=4-7 mice in each). On day 0, animals underwent bile duct ligation. From day 7, animals were treated for five days with vehicle or different doses of Compound 1, as shown in Table 2. Compound 1 was administered orally by gavage twice per day. Spontaneous urine samples were collected before BDL and on days 7, 8, 9, 10, 11 and 12 after BDL by cannulation of the urinary bladder, and the concentration of bile acids in urine was determined.

TABLE 2

| Group | | | Dose | Volume | | Number of |
|---|---|---|---|---|---|---|
| No. | Test item | Frequency | (mg/kg) | (mL/kg) | Route | animals |
| 1 | Vehicle | Twice daily | 0 | 4 | p.o. | 4 |
| | | for 5 days | | | | |
| | | (day 7-11) | | | | |
| 2 | Com- | Twice daily | 15 | 4 | p.o. | 4 |
| | pound 1 | for 5 days | | | | |
| | | (day 7-11) | | | | |
| 3 | Com- | Twice daily | 30 | 4 | p.o. | 6 |
| | pound 1 | for 5 days | | | | |
| | | (day 7-11) | | | | |
| 4 | Com- | Twice daily | 60 | 4 | p.o. | 5 |
| | pound 1 | for 5 days | | | | |
| | | (day 7-11) | | | | |
| 5 | Com- | Twice daily | 120 | 4 | p.o. | 5 |
| | pound 1 | for 5 days | | | | |
| | | (day 7-11) | | | | |

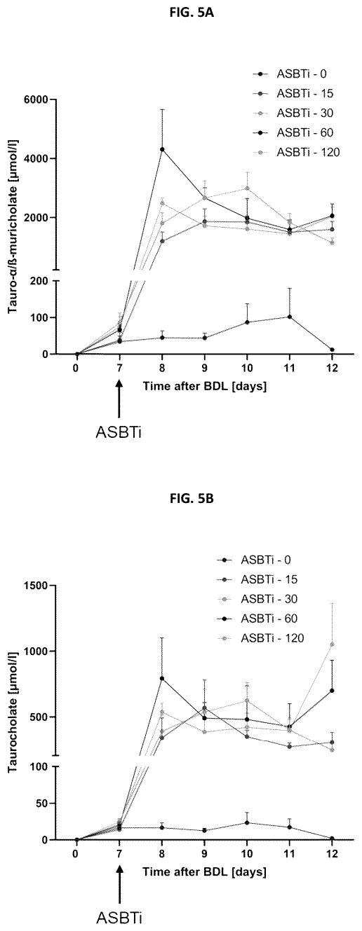

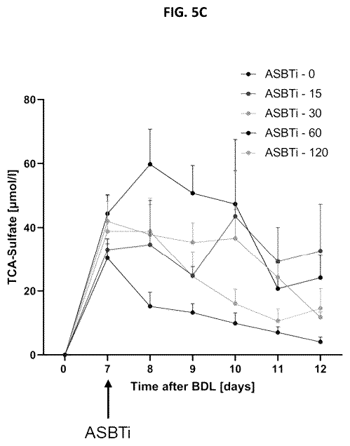

The dose-dependent study demonstrated strongly increased urinary bile acid due to ASBT inhibition during the 5-days analysis period. The urinary excretion of bile acids was drastically increased following treatment with Compound 1, with concentrations of bile acids being at least about 20-fold higher in all treated groups. The results are shown in FIGS. 4 (the total concentration of all bile acids) and FIGS. 5A-5C (the concentrations of the individual bile acids tauro-α/β-muricholate, taurocholate and taurocholic acid sulfate, respectively).

### Example 3

### Effect of ASBT Inhibition on Cholemic Nephropathy in Bile Duct Ligated Mice

The long-term effect of Compound 1 on kidney injury in BDL mice was studied by histology, clinical chemistry and intravital imaging.

Female mice (C57BL/6n) were used. After completion of quarantine and acclimatization period, animals were randomized based on body weight into experimental groups (n=15 mice per group). On day 0, animals underwent bile duct ligation or sham surgery. Animals were treated with vehicle or with Compound 1 at a dose of 60 mg/kg, as shown in Table 3. Compound 1 was administered orally by gavage twice per day. Spontaneous urine samples were collected before BDL as well as on day 1 and weeks 1, 2, 3, 4, 5, and 6 after BDL by cannulation of the urinary bladder. Blood, urine, bile and tissue samples were collected at the end (week 6) of the experiment.

TABLE 3

| Group | | | | Dose | Volume | | Number of |
|---|---|---|---|---|---|---|---|
| No. | Surgery | Test item | Frequency | (mg/kg) | (mL/kg) | Route | animals |
| 1 | Sham | Vehicle | Twice daily | 0 | 4 | p.o. | 15 |
| | | | for 6 weeks | | | | |
| 2 | BDL | Vehicle | Twice daily | 0 | 4 | p.o. | 15 |
| | | | for 6 weeks | | | | |
| 3 | BDL | Com- | Twice daily | 60 | 4 | p.o. | 15 |
| | | pound 1 | for 6 weeks | | | | |

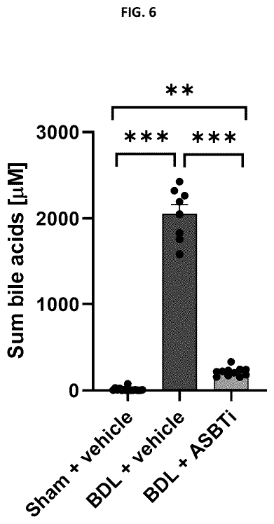

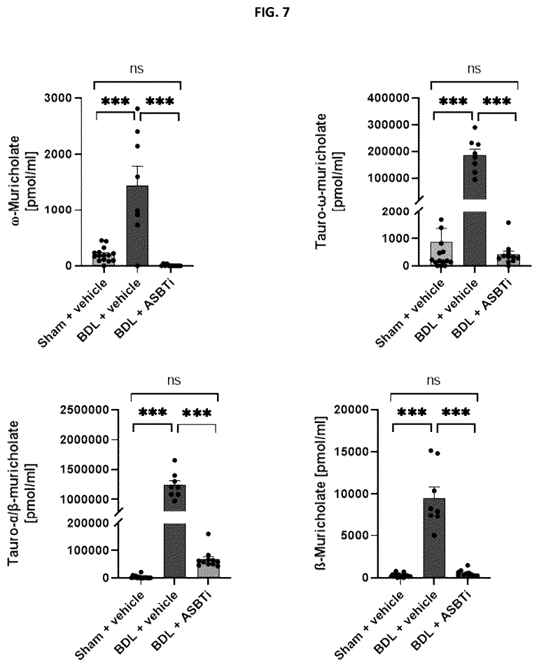

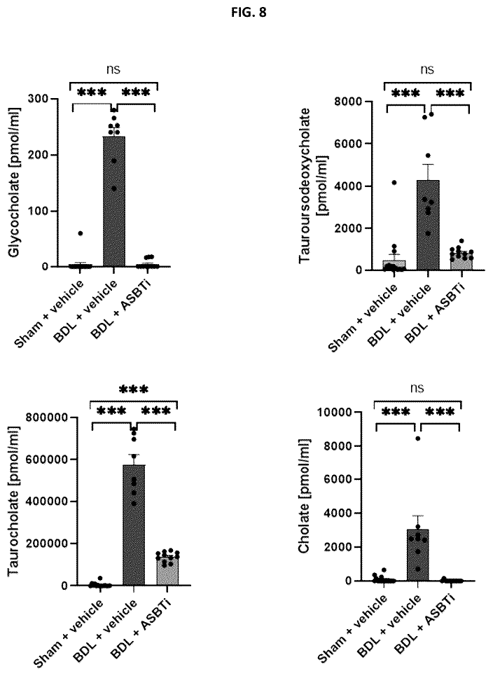

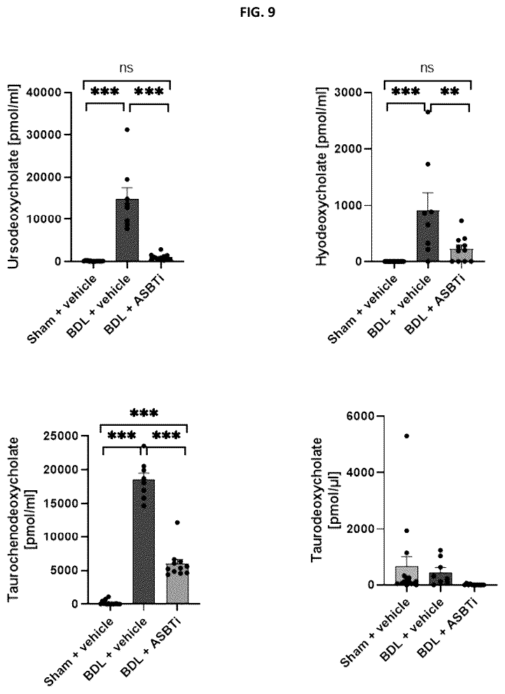

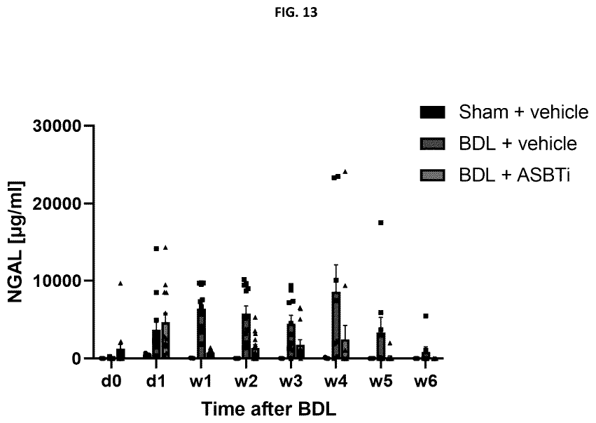

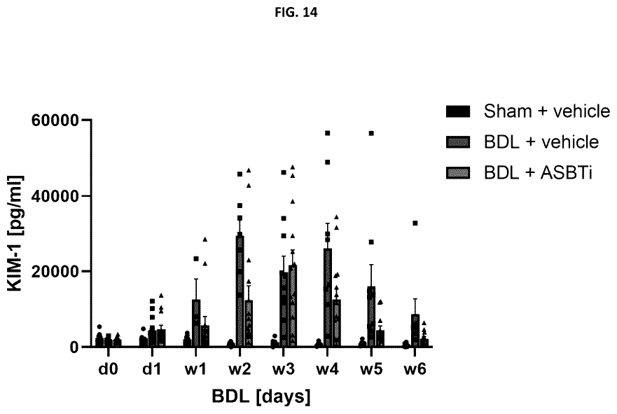

Severe fibrosis and cystic dilatation of renal tubules was seen in the BDL vehicle controls which was antagonized by Compound 1 almost to the levels of control animals without BDL. The urinary kidney damage markers Neutrophil Gelatinase-Associated Lipocalin (NGAL) and Kidney Injury Molecule-1 (KIM-1) were strongly reduced by Compound 1 (see FIGS. 13 and 14, respectively). Additionally, serum BA decreased by about 90% (See FIG. 6 for total bile acids, and FIGS. 7-10 for individual bile acids) and urinary BA massively increased. Intravital imaging with Evans blue demonstrated severe vascular leakage in kidneys after BDL, which was completely reversed by Compound 1.

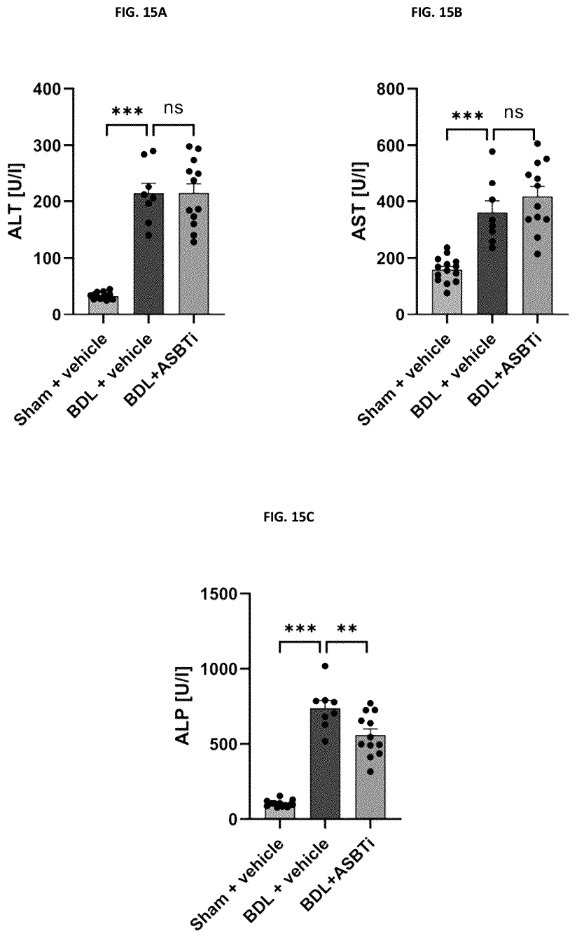

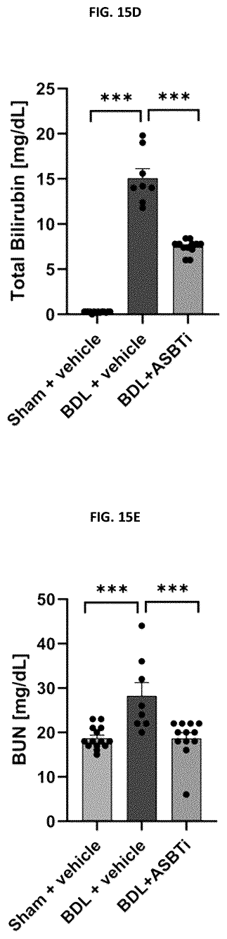

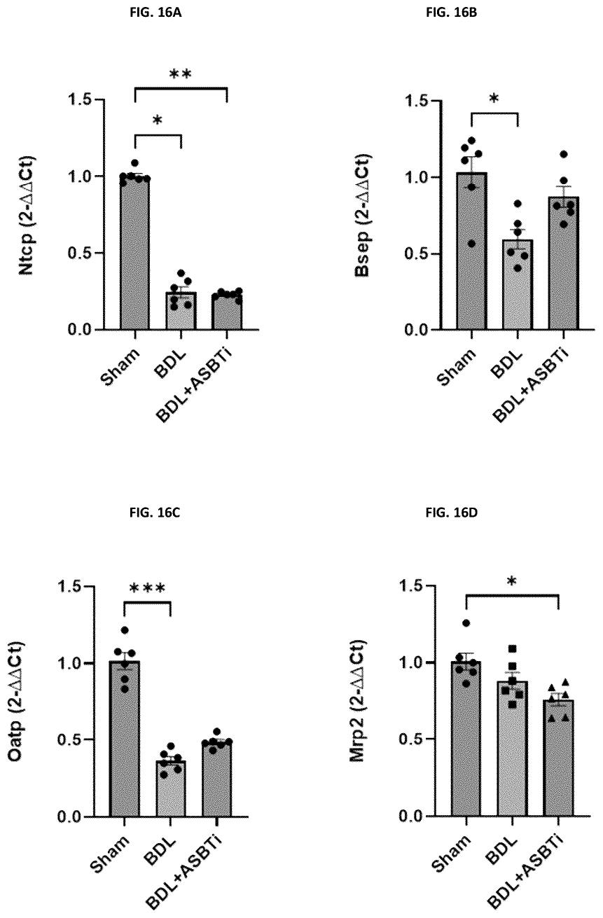

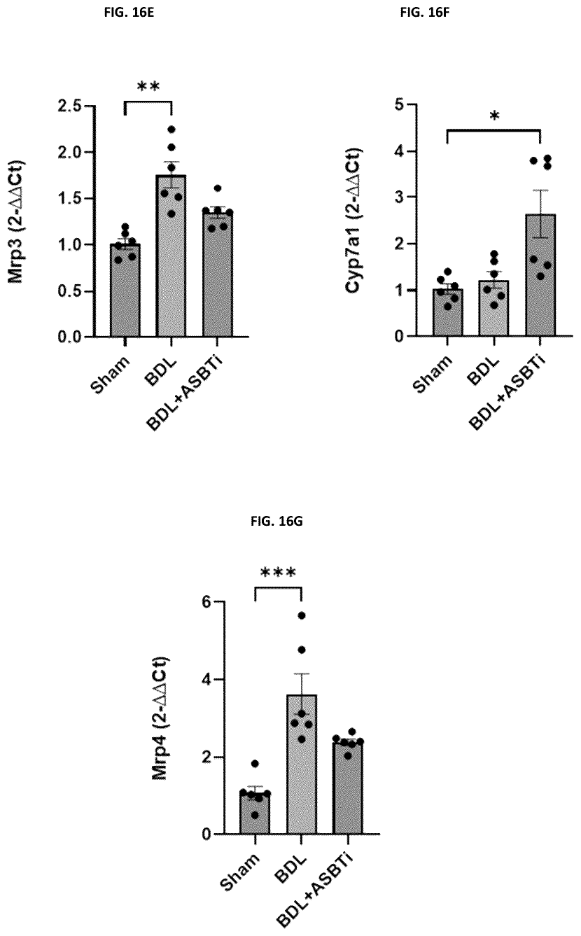

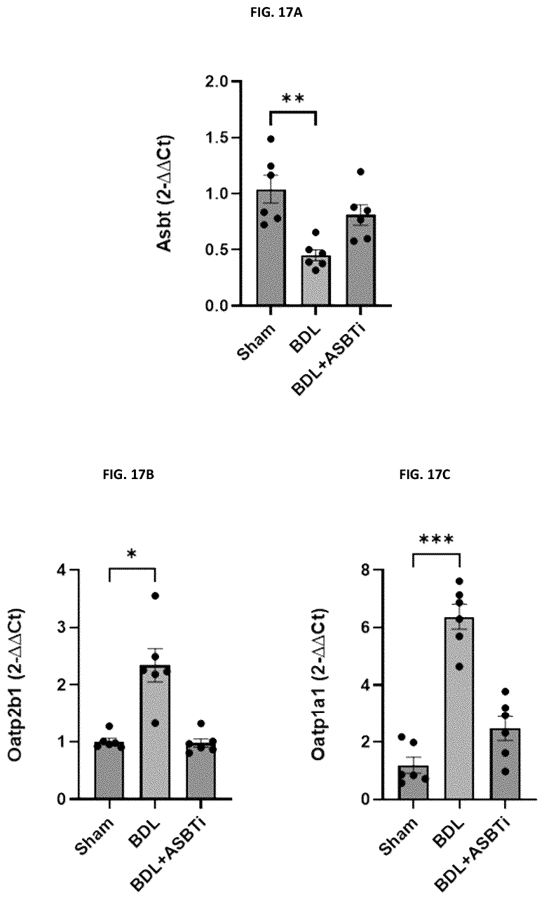

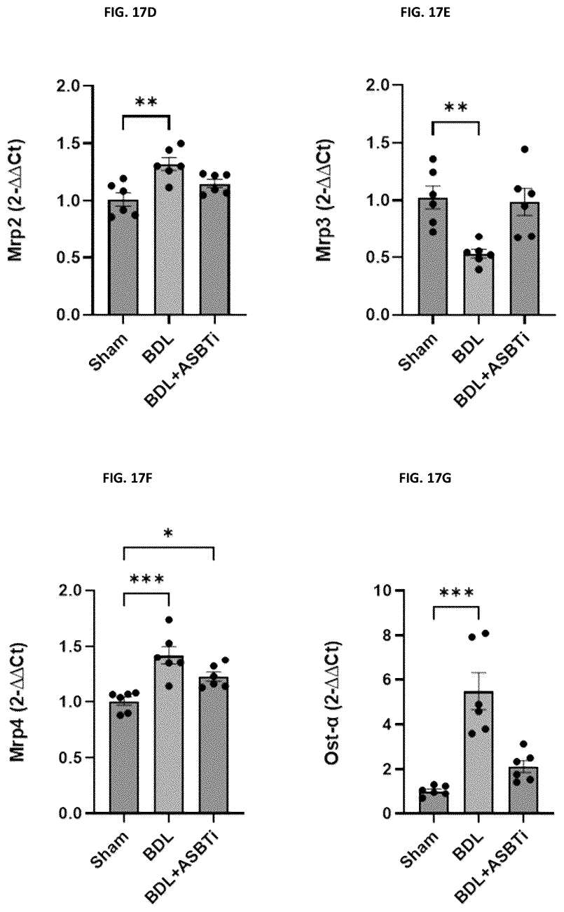

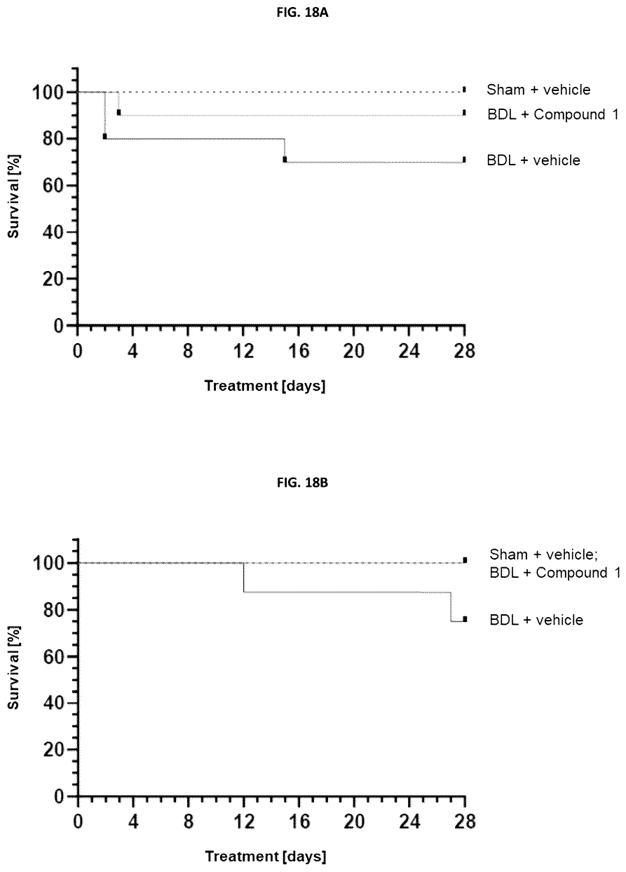

The levels of liver enzymes (ALT, AST and ALP), total bilirubin and blood urea nitrogen (BUN) following treatment for 6 weeks are shown in FIGS. 15A-15E. The expression of different bile acid transporters following treatment for 6 weeks is shown in FIGS. 16A-16G and 17A-17G.

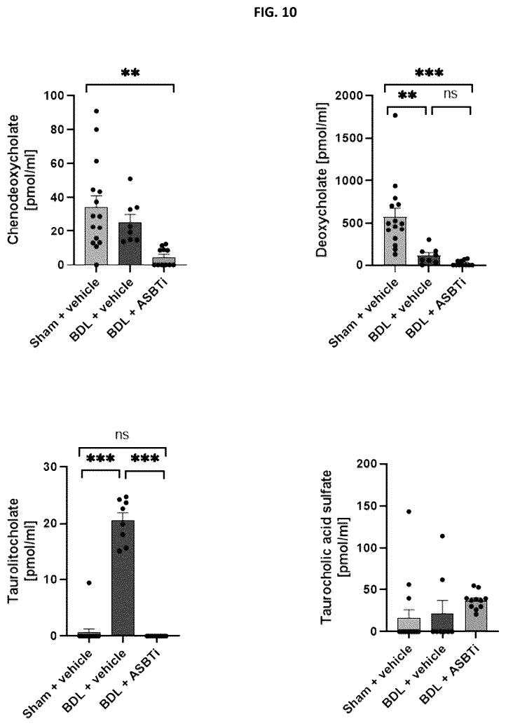

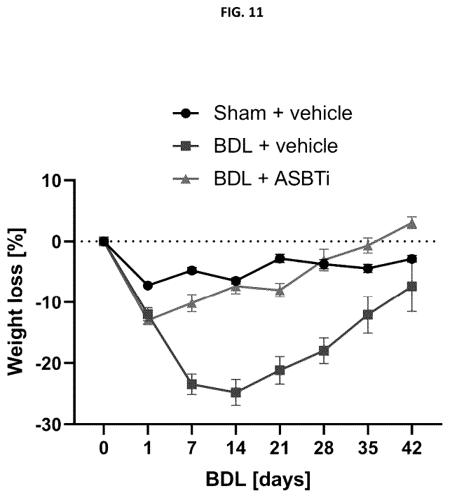

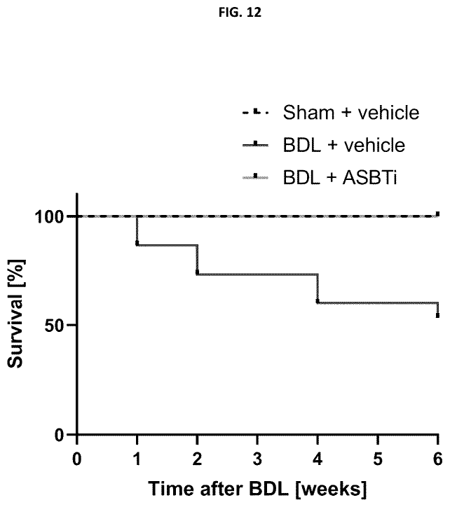

In the BDL-vehicle group about 50% of the mice died in the 6 week-period, while no mortality occurred in the group of BDL mice treated with Compound 1 (see FIG. 12). Also, the body weight loss due to BDL was strongly ameliorated by ASBT inhibition (see FIG. 11). A similarly designed study with male mice (6 per group) confirmed the therapeutic efficacy of Compound 1 observed in the female cohort.

### Example 4

### Investigation of Therapeutic Time Window

The effect of the time period before start of treatment with Compound 1 on kidney injury in BDL mice was evaluated.

Male mice (C57BL/6n) were used. After completion of quarantine and acclimatization period, animals were randomized based on body weight into experimental groups (n=7 mice per group). On day 0, animals underwent bile duct ligation or sham surgery. From day 3, 21, 42 or 63, animals were treated for 28 days with either vehicle or with Compound 1 at a dose of 60 mg/kg, as shown in Table 4 below. Compound 1 was administered p.o. twice per day. The animals were weighed every day. Blood, urine, bile and tissue samples were collected at the end of the experiment.

TABLE 4

| Group | Subgroup | | | Start of | Number of |
|---|---|---|---|---|---|
| No. | No. | Surgery | Test item | treatment | animals |
| 1 | 1 | Sham | Vehicle | Day 3 | 7 |
| | 2 | BDL | Vehicle | Day 3 | 7 |
| | 3 | BDL | Compound 1 | Day 3 | 7 |
| 2 | 1 | Sham | Vehicle | Day 21 | 7 |
| | 2 | BDL | Vehicle | Day 21 | 7 |
| | 3 | BDL | Compound 1 | Day 21 | 7 |
| 3 | 1 | Sham | Vehicle | Day 42 | 7 |
| | 2 | BDL | Vehicle | Day 42 | 7 |
| | 3 | BDL | Compound 1 | Day 42 | 7 |
| 4 | 1 | Sham | Vehicle | Day 63 | 7 |
| | 2 | BDL | Vehicle | Day 63 | 7 |
| | 3 | BDL | Compound 1 | Day 63 | 7 |

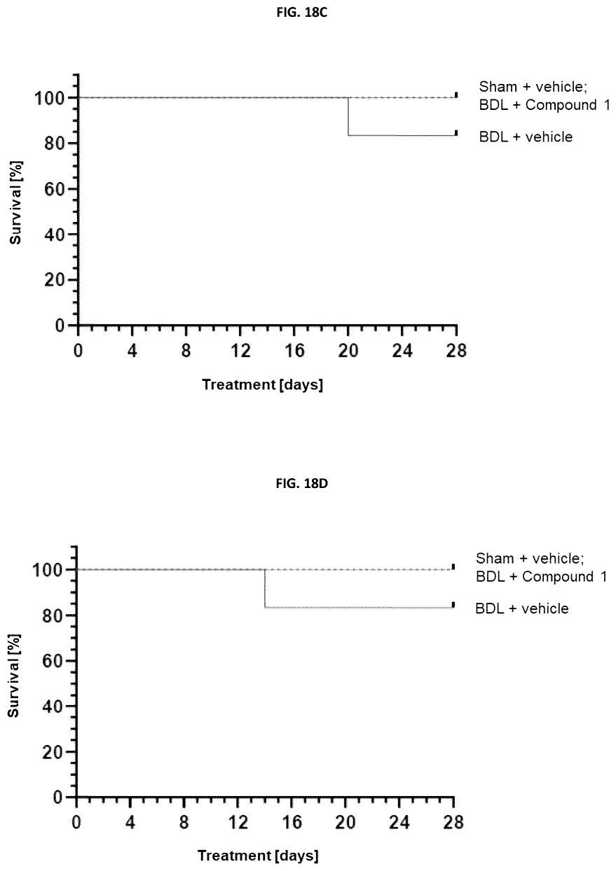

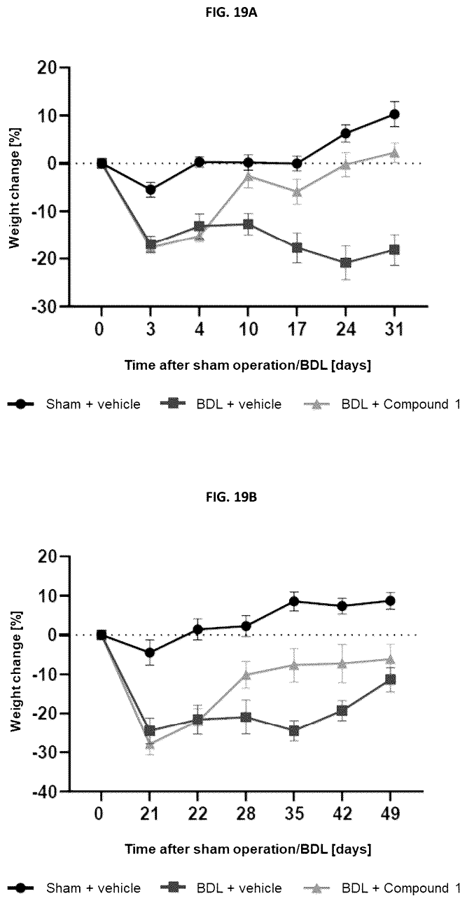

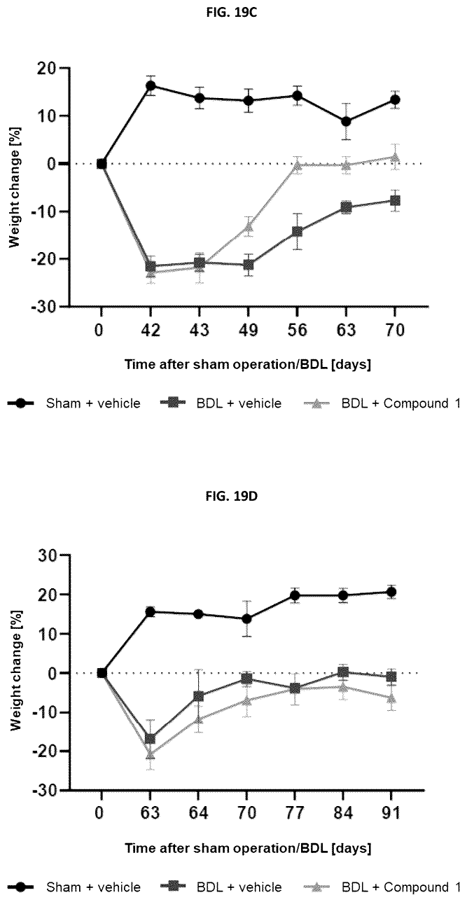

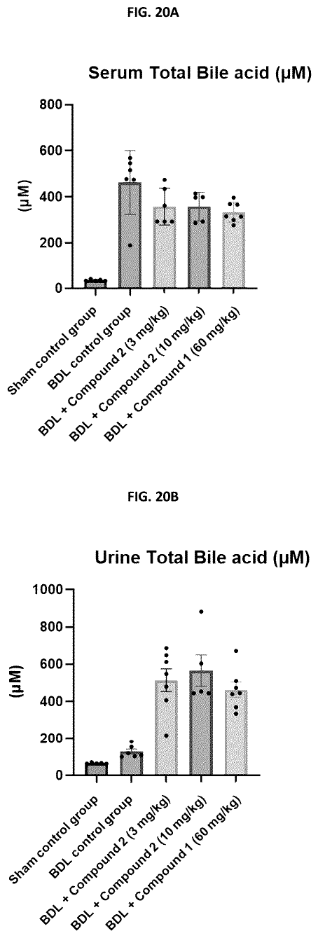

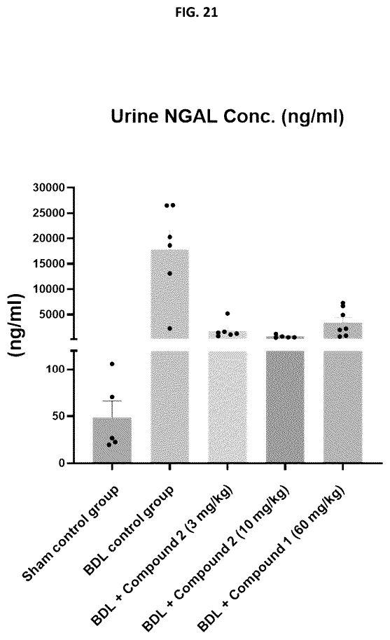

Plots of the survival rates (%) for the four groups are shown in FIGS. 18A-18D. Plots of the weight change (%) for the four groups after sham operation or BDL are shown in FIGS. 19A-19D.

4-week treatment with Compound 1 completely prevented renal injury and reversed body weight loss when dosing started on Day 3. Compound 1 almost completely reversed renal injury and reversed body weight loss when dosing started on Day 21. Compound 1 partially reversed renal injury and reversed body weight loss when dosing started on Day 42. Compound 1 had minimal effects when dosing started on Day 63.

### Example 5

### Comparison of Different ASBT Inhibitors

Male mice (C57BL/6n) were used. After completion of quarantine and acclimatization period, animals were randomized based on body weight into experimental groups (n=12 mice per bile duct ligation group along with 5 sham operated controls). On day 0, animals underwent bile duct ligation or sham surgery. From day 3, animals were treated for 19 days with vehicle or an ASBT inhibitor as shown in Table 5 below. The ASBT inhibitors were administered p.o. once daily. The animals were weighed every day. At day 21, blood samples were taken 2 and 6 hours after the final dose. At day 24, animals were sacrificed and samples (urine and blood; liver, kidney, heart and spleen tissues) were collected and analysed.

TABLE 5

| Group | | | Dose | Number of |
|---|---|---|---|---|
| No. | Surgery | Test item | (mg/kg) | animals |
| 1 | Sham | Vehicle | 0 | 5 |
| 2 | BDL | Vehicle | 0 | 12 |
| 3 | BDL | Compound 1 | 60 | 12 |
| 4 | BDL | Compound 2 | 3 | 12 |
| 5 | BDL | Compound 2 | 10 | 12 |

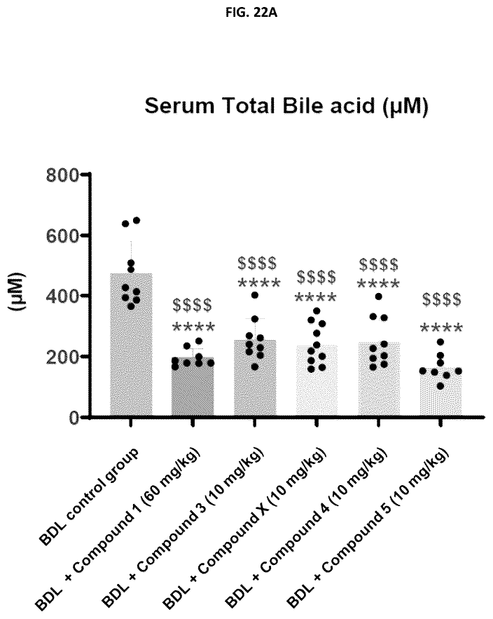

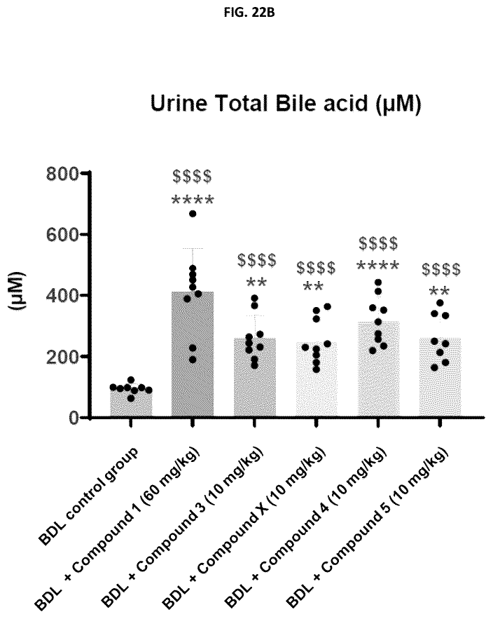

Compounds 1 and 2 were observed to lower serum bile acids and to increase urine bile acids after treatment for 19 days. The concentration of total bile acids in blood and urine at day 21 (i.e., day 19 of treatment) is shown in FIGS. 20A and 20B, respectively. Compounds 1 and 2 also lowered urinary NGAL after treatment for 18 days; see FIG. 21.

### Example 6

### Comparison of Different ASBT Inhibitors

Male mice (C57BL/6n) were used. After completion of quarantine and acclimatization period, animals were randomized based on body weight into experimental groups (n=10 mice per bile duct ligation group). On day 1, animals underwent bile duct ligation. From day 4, animals were treated for 5 days with vehicle or an ASBT inhibitor, as shown in Table 6 below. The ASBT inhibitors were administered p.o. once daily. The animals were weighed every day. At day 6, animals were moved to individual cages for cumulative overnight fecal collection (day 6 to 7). At day 8, blood samples were taken 2 and 6 hours after the final dose. Animals were then sacrificed and samples (urine and blood; liver, kidney and ileum tissues) were collected and analysed.

TABLE 6

| Group | | | Dose | Number of |
|---|---|---|---|---|
| No. | Surgery | Test item | (mg/kg) | animals |
| 1 | BDL | Vehicle | 0 | 10 |
| 2 | BDL | Compound 1 | 60 | 10 |
| 3 | BDL | Compound 3 | 10 | 10 |
| 4 | BDL | Compound X | 10 | 10 |
| | | (undisclosed ASBT inhibitor) | | |
| 5 | BDL | Compound 4 | 10 | 10 |
| 6 | BDL | Compound 5 | 10 | 10 |

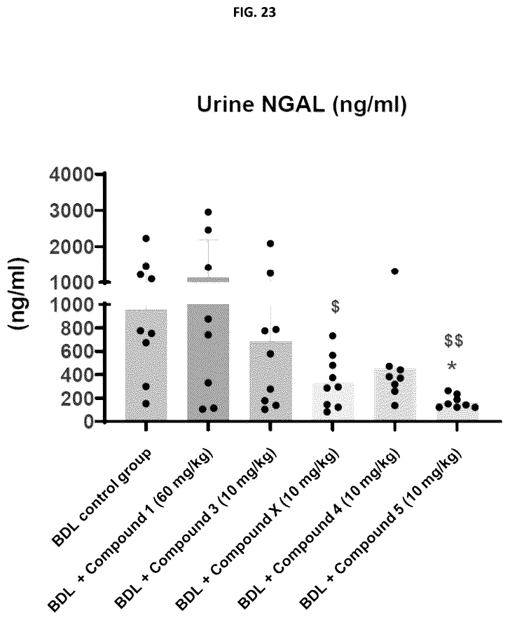

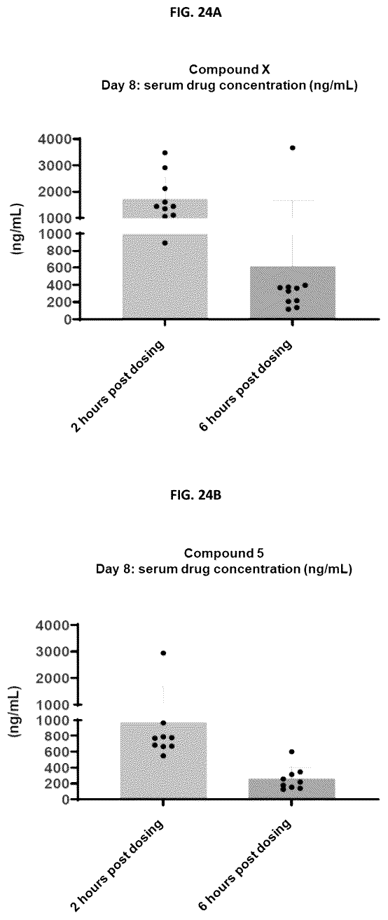

All compounds were observed to lower serum bile acids and to increase urine bile acids after treatment for 5 days. The concentration of total bile acids in blood and urine at day 8 (i.e., day 5 of treatment) is shown in FIGS. 22A and 22B, respectively. Compound X and Compound 5 significantly reduced urinary NGAL after treatment for 5 days; see FIG. 23.

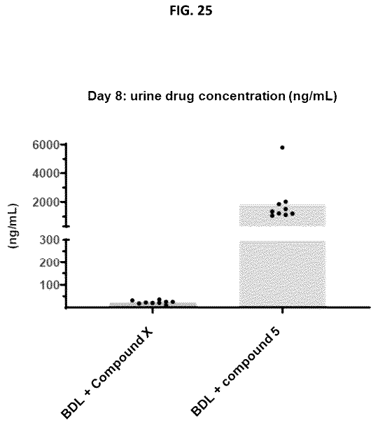

The concentrations of Compound X and Compound 5 at day 8 in serum (at 2 and 6 hours post dosing) and in urine (up to 6 hours post dosing) are shown in FIGS. 24 and 25, respectively.

1. (canceled)

2. The method according to claim 25, wherein the renal disease or disorder is a bile acid dependent renal disease or disorder.

3. The method according to claim 25, wherein the renal disease or disorder is selected from the group consisting of cholemic nephropathy, chronic nephropathy, hyperbilirubinemia, renal dysfunction of obstructive jaundice, aging-induced impaired mitochondrial functions in the kidney, renal inflammation, acute kidney injury (AKI), kidney ischemia/reperfusion injury (IRI), chronic kidney disease (CKD), chronic renal insufficiency, end-stage renal disease (ESRD), proximal tubule damage in the kidney, hepatorenal syndrome type 1, hepatorenal syndrome type 2, and acute-on-chronic liver disease.

4. The method according to claim 25, wherein the renal disease or disorder is cholemic nephropathy.

5. The method according to claim 25, wherein the ASBT inhibitor is (Z)-3-((3-butyl-3-ethyl-7-(methylthio)-1,1-dioxido-5-phenyl-2,3,4,5-tetrahydro-1,5-benzothiazepin-8-yl)oxy)-2-fluoroacrylic acid, or a pharmaceutically acceptable salt thereof.

6. The method according to claim 25, wherein the ASBT inhibitor is selected from the group consisting of elobixibat, odevixibat, maralixibat, volixibat and linerixibat, or a pharmaceutically acceptable salt thereof.

7. The method according to claim 25, wherein the ASBT inhibitor is administered orally.

8. The method according to claim 25, wherein the systemic absorption of the ASBT inhibitor is greater than 10%.

9. The method according to claim 25, wherein the ASBT inhibitor is administered subcutaneously.

10. The method according to claim 9, wherein the method further comprises orally administering to the subject a non-systemically available ASBT inhibitor.

11. The method according to claim 25, wherein the subject exhibits a reduction in serum bile acid concentration following administration of the ASBT inhibitor.

12. The method according to claim 11, wherein the reduction in serum bile acid concentration is at least 60%, at least 70%, at least 80% or at least 90% relative to baseline.

13. The method according to claim 11, wherein the serum bile acid concentration is normalized following administration of the ASBT inhibitor.

14. The method according to claim 25, wherein the subject exhibits an increase in urinary bile acids following administration of the ASBT inhibitor.

15. The method according to claim 14, wherein the increase in urinary bile acids is at least 60% relative to baseline.

16. The method according to claim 25, wherein the subject exhibits an improvement in one or more liver parameters following administration of the ASBT inhibitor.

17. The method according to claim 16, wherein the one or more liver parameters are selected from the group consisting of serum total bilirubin level, serum alkaline phosphatase (ALP) level, serum alanine aminotransferase (ALT) level and serum aspartate aminotransferase (AST) level.

18. The method according to claim 16, wherein the improvement in the one or more liver parameters occurs following administration of the ASBT inhibitor for at least 4 weeks.

19. The method according to claim 25, wherein the subject exhibits a reduction in urinary neutrophil gelatinase-associated lipocalin (NGAL) following administration of the ASBT inhibitor.

20. The method according to claim 19, wherein the reduction in urinary NGAL is at least 60%.

21. The method according to claim 25, wherein the subject exhibits a reduction in urinary kidney injury molecule-1 (KIM-1) following administration of the ASBT inhibitor.

22. The method according to claim 21, wherein the reduction in urinary KIM-1 is at least 60%.

23. The method according to claim 25, wherein the subject exhibits a reduction in serum blood urea nitrogen (BUN) following administration of the ASBT inhibitor.

24. The method according to claim 25, wherein the ASBT inhibitor is administered once daily.

25. A method of treating a renal disease or disorder in a subject, the method comprising administering to the subject a therapeutically effective amount of an ASBT inhibitor, or a pharmaceutically acceptable salt thereof.
